(19) United States
(12) Patent Application Publication
(10) Pub. No.: US 2026/0027380 A1
(43) Pub. Date: Jan. 29, 2026

(54) TECHNIQUES FOR MAGNETIC NANOCLUSTER-BASED COMBINATION THERAPY

(71) Applicant: William Marsh Rice University, Houston, TX (US)

(72) Inventors: Gang Bao, Houston, TX (US); Linlin Zhang, Houston, TX (US); Qingbo Zhang, Houston, TX (US)

(21) Appl. No.: 18/997,657

(22) PCT Filed: Jul. 26, 2023
(86) PCT No.: PCT/US23/28722

§ 371 Date: Jan. 22, 2025

Related U.S. Application Data
(60) Provisional application No. 63/392,690, filed on Jul. 27, 2022.

(51) Int. Cl. A61N 2/00; A61K 9/00; A61K 31/655; A61K 45/06; A61K 47/02; A61K 47/69; A61P 35/00

(52) U.S. Cl. CPC A61N 2/004; A61K 9/0019; A61K 31/655; A61K 45/06; A61K 47/02; A61K 47/6929; A61N 2/002; A61P 35/00

(57) ABSTRACT

The present disclosure relates to treating tumor tissue using magneto-immunotherapy (Mag-IT) techniques. For example, a method may include administering a magnetotherapy treatment to tumor tissue of a subject and administering an immune checkpoint blockade (ICB) treatment to the subject. The magnetotherapy treatment may include positioning a nanoplatform adjacent to or in contact with the tumor tissue and applying a magnetic field to the nanoplatform.

### CROSS-REFERENCE TO RELATED APPLICATIONS

This application claims priority to PCT application No. PCT/US23/28722, entitled “TECHNIQUES FOR MAGNETIC NANOCLUSTER-BASED COMBINATION THERAPY,” filed Jul. 26, 2023, which claims benefit of U.S. Provisional Application No. 63/392,690, entitled “TECHNIQUES FOR MAGNETIC NANOCLUSTER-BASED COMBINATION THERAPY”, filed Jul. 27, 2022, which are incorporated by reference in all their entirety herein.

### GOVERNMENT LICENSE RIGHTS

This invention was made with government support under RR140081 awarded by the Cancer Prevention and Research Institute of Texas (CPRIT). The government has certain rights in the invention.

### FIELD OF THE TECHNOLOGY

The subject matter disclosed herein relates to techniques for magnetic nanocluster-based combination therapy for cancer treatment.

### BACKGROUND

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Cancer remains a leading cause of death worldwide. For instance, in 2020, cancer accounted for approximately ten million deaths according to the World Health Organization (WHO). The main causes for cancer-related deaths are recurrence and metastasis of cancer after the initial treatment of primary tumors. Conventional cancer therapeutics, such as surgery, chemotherapy, and radiation therapy, have shown limited success in preventing cancer relapse and metastasis. However, the emergence and development of immunotherapy offers the possibility for long-term control of cancer. In particular, immune checkpoint blockade (ICB) therapy is a promising immunotherapy that has been approved for clinical use in treating several types of late-stage solid tumors. However, the overall response rates to ICB therapy of solid tumors are low due to various reasons, such as low immunogenicity of tumors, lack of tumor-infiltrating immune cells, and an immunosuppressive tumor microenvironment (TME). Accordingly, it may be desirable for improved techniques for providing ICB therapy for treating cancer and/or tumor tissue.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

The present disclosure is directed to treating cancer and/or tumor tissue using magneto-immunotherapy (Mag-IT) techniques. In particular, the techniques described herein combine magnetic nanocluster-based hyperthermia, free radical generation, and immune checkpoint blockade (ICB) therapy to induce immunogenic cell death (ICD) of tumor tissue of a subject so as to provide effective suppression of both primary and secondary tumors. In certain embodiments, a nanoplatform that generates localized heat and free radicals under an alternating magnetic field (AMF) may be positioned within a region that includes the tumor tissue of the subject or is near or adjacent to the tumor tissue of the subject. Specifically, the nanoplatform may include one or more iron oxide nanocrystal clusters (IONCs) and one or more 2,2′-Azobis (2-midinopropane) dihydrochloride (AAPH) molecules attached so as to form an IONC-AAPH nanoplatform. After applying the AMF to the region including the nanoplatform, the nanoplatform simultaneously generates local heat and free radicals that induce ICD in the tumor tissue of the subject under both normoxic and hypoxic conditions. In this way, the nanoplatform may efficiently eradicate the tumor tissue in the subject. Additionally, combining treatment of the tumor tissue with the nanoplatform with ICB therapy may trigger the abscopal effect and immune memory.

In particular, the tumor cell death caused by the combination of magnetic heating and free radicals causes the release or exposure of various damage-associated molecule patterns, which promote the maturation of dendritic cells. In studies performed and discussed herein, treating tumor-bearing mice with IONC-AAPH under AMF not only eradicated the tumors but also generated systemic antitumor immune responses. The combination of IONC-AAPH under AMF with anti-PD-1 ICB dramatically suppressed the growth of untreated distant tumors and induced long-term immune memory. This IONC-AAPH based magneto-immunotherapy has the potential to effectively combat metastasis and control cancer recurrence, thereby providing improved techniques for controlling the metastasis and/or relapse of cancer.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic illustration of heat and free radical generation from an iron oxide nanocrystal cluster (IONC)-2,2′-Azobis (2-midinopropane) dihydrochloride (AAPH) nanoplatform under an alternating magnetic field (AMF), in accordance with aspects of the present approach;

FIG. 2 is a magnetization curve that shows negligible coercivity of IONC-AAPH, in accordance with aspects of the present approach;

FIG. 3 illustrates the quantification of ABTS+· absorbance under various conditions, in accordance with aspects of the present approach;

FIG. 4 illustrates a flow cytometry analysis of intracellular free radicals in MC-38 cells detected via fluorescence from DCF, in accordance with aspects of the present approach;

FIG. 5 is a graph illustrating the quantification of intracellular free radicals, in accordance with aspects of the present approach;

FIG. 6 illustrates a flow cytometry analysis of cell death in MC-38 cells after different treatment under normoxic and hypoxic conditions, in accordance with aspects of the present approach;

FIG. 7 illustrates a quantification of γ-H2AX fluorescence, in accordance with aspects of the present approach;

FIG. 8 illustrates a quantification of MC-38 cells with F-actin structure in the morphological analysis, in accordance with aspects of the present approach;

FIG. 9 illustrates a flow cytometry analysis of mitochondrial membrane potential of MC-38 cells four hours after treatment by JC-1 staining, in accordance with aspects of the present approach;

FIG. 10 illustrates a quantification of the J-aggregate to J-monomer ratio using the mean fluorescence detected by the flow cytometry analysis, in accordance with aspects of the present approach;

FIG. 11 illustrates a glutathione (GSH) depletion measurement of MC-38 cells two hours after treatment, in accordance with aspects of the present approach;

FIG. 12 illustrates a quantification of malondialdehyde (MDA) levels in MC-38 cells two hours after treatment, in accordance with aspects of the present approach;

FIG. 13 illustrates quantification of DC maturation using representative flow cytometry data, in accordance with aspects of the present approach;

FIG. 14 illustrates the quantification data of FIG. 13, in accordance with aspects of the present approach;

FIG. 15 illustrates tumor growth curves of different treatment groups, in accordance with aspects of the present approach;

FIG. 16 illustrates weights of excised tumors, in accordance with aspects of the present approach;

FIG. 17 illustrates the tumor growth curves of the individual mice, in accordance with aspects of the present approach;

FIG. 18 illustrates the average growth curve of secondary tumors, in accordance with aspects of the present approach;

FIG. 19 illustrates individual tumor growth, in accordance with aspects of the present approach;

FIG. 20 illustrates survival curves of mice receiving different treatments, in accordance with aspects of the present approach;

FIG. 21 illustrates the representative gating strategy for analyzing tumor infiltrating lymphocytes in secondary tumors, in accordance with aspects of the present approach;

FIG. 22 illustrates flow cytometry data representative of infiltrating immune cells in secondary tumors, in accordance with aspects of the present approach;

FIG. 23 illustrates the corresponding quantification data for FIG. 22, in accordance with aspects of the present approach;

FIG. 24 depicts quantification data in which tumor antigen-specific and IFN-γ secreting T cells were detected, in accordance with aspects of the present approach;

FIG. 25 is a graph illustrating the body weights of mice recorded daily over time, in accordance with aspects of the present approach;

FIG. 26 illustrates respective heating curves of three types of MIONs and water, in accordance with aspects of the present approach;

FIG. 27 illustrates the heating efficiency (characterized as the specific absorption rate (SAR) of the MIONs of FIG. 26, in accordance with aspects of the present approach;

FIGS. 28A and 28B in combination illustrate that murine pancreatic cancer cell line mT5 can be killed by MIONs under AMF in a concentration-dependent manner, in accordance with aspects of the present approach;

FIG. 29 illustrates heating curves for 40 nm IONCs, in accordance with aspects of the present approach;

FIG. 30 is a plot of tumor size post treatment with and without diffusion, in accordance with aspects of the present approach;

FIG. 31 illustrates quantitative results of a study of the biodistribution of IONCs at 24 hours after intratumoral injection of clusters, in accordance with aspects of the present approach;

FIG. 32 illustrates quantitative results of a study of the biodistribution of IONCs at 16 days after intratumoral injection of clusters, in accordance with aspects of the present approach;

FIG. 33 depicts results of a rechallenge study as pertains to central memory T cells, in accordance with aspects of the present approach;

FIG. 34 depicts results of a rechallenge study as pertains to effector memory T cells, in accordance with aspects of the present approach; and

FIG. 35 schematically illustrates a gating strategy employed for the analysis of tumor-specific memory T cells, in accordance with aspects of the present approach.

### DETAILED DESCRIPTION

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. One or more specific implementations will be described below. In an effort to provide a concise description of these implementations, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As mentioned above, immune checkpoint blockade (ICB) therapy is a promising immunotherapy that has been approved for clinical use in treating several types of late-stage solid tumors. However, the overall response rates to ICB therapy are low due to various reasons, such as low immunogenicity of tumors, lack of tumor-infiltrating immune cells, and an immunosuppressive tumor microenvironment (TME). Combining ICB with other cancer treatment modalities that can modulate tumor immunogenicity has shown great promise in improving ICB therapy. Several cancer treatment modalities have been found to cause immunogenic cell death (ICD), thus increasing the immunogenicity of tumor tissue and activating anti-tumor immunity. Among these approaches, local hyperthermia offers great therapeutic advantages due to minimal invasiveness and low side effects. For example, photothermal therapy is one of the most efficient methods to deliver local heat to tumor tissue. However, the limited penetration depth of light in biological tissues restricts the application of photothermal therapy in treating deep-seated tumors.

Magnetic hyperthermia provides an attractive approach for cancer treatment because a magnetic field has an unlimited penetration depth in biological tissues. However, one major challenge for magnetic hyperthermia is a low magneto-thermal efficiency of iron oxide nanoparticles. Additionally, free radicals have demonstrated great potential in modulating tumor immunogenicity. Free radicals, such as reactive oxygen species (ROS), produced by radiation therapy, photodynamic therapy, and sonodynamic therapy can cause ICD, thus priming anti-tumor immune responses. However, the production of ROS from these therapeutic modalities may be hindered by low oxygen levels in the TME, limiting the effectiveness of these treatments. Various adjunctive strategies have been developed to overcome a hypoxic TME, such as combining ROS-inducing agents with hypoxia-activated prodrugs and reversing tumor hypoxia with oxygen carriers. However, these methods suffer from the complexity in designing integrated therapeutic agents, a low co-delivery efficiency, and other adverse effects. Further, azo compounds integrated with photothermal agents can generate oxygen-independent free radicals under a beam laser. However, this approach is limited by the penetration depth of light in biological tissues.

Accordingly, the present disclosure is directed to treating cancer and/or tumor tissue using magneto-immunotherapy (Mag-IT) techniques. In particular, the techniques described herein combine magnetic hyperthermia, free radical generation, and immune checkpoint blockade (ICB) therapy to induce immunogenic cell death (ICD) of tumor tissue of a subject. In certain embodiments, a nanoplatform that generates localized heat and free radicals under an alternating magnetic field (AMF) may be positioned within a region that includes the tumor tissue of the subject or is near or adjacent to the tumor tissue of the subject. As discussed herein, upon heating an azo compound decomposes to generate free radicals quickly and without the need of oxygen. As described, magnetic iron oxide nanocrystal clusters (IONC) were loaded with water-soluble azo compound 2,2′-azobis(2-amidinopropane) dihydrochloride (AAPH), which decomposes by heat generated from IONC under an applied alternating magnetic field (AMF), resulting in carbon-centered free radicals. Free radicals generated from the decomposition of AAPH can sensitize cancer cells to heat and enhance heat-induced cancer cell killing. The simultaneous generation of localized heat and free radicals can be used to induce immunogenic cell death and efficiently eradicate the primary tumors at low dose of nontoxic IONCs under the clinically safe AMF. When the use of local heat and free radical generation is combined with ICB, the resulting magneto-immunotherapy demonstrates efficient abscopal effect and long-term immune memory, demonstrating potential for improving ICB therapy and suppressing tumor recurrence and metastasis. Compared with light-based methods, such as photothermal therapy and photodynamic therapy, the magneto-immunotherapy discussed herein can be used to treat primary and distant tumors in deep tissue since a magnetic field can be applied to the whole body.

With the preceding in mind, in certain embodiments, the nanoplatform may be synthesized from an IONC and one or more AAPH molecules. For instance, the IONC may be synthesized through metal salt hydrolysis, such as iron salt hydrolysis. After synthesizing the IONC, the IONC may be coated with polyacrylic acid (PAA). The surface of the PAA-coated IONC may then be modified with poly(AA-co-AMPS-co-PEG). After modifying the surface of the PAA-coated IONC with poly(AA-co-AMPS-co-PEG), a PAA on the surface of the IONC may bond with a nitrodopamine molecule. Thereafter, an AAPH molecule may conjugate with the nitrodopamine-PAA chain (e.g., a polymer chain) on the surface of the IONC, thereby resulting in the IONC-AAPH nanoplatform.

After one or more IONC-AAPH nanoplatforms have been synthesized, the IONC-AAPH nanoplatforms may be administered to a subject having a tumor. For instance, the tumor may be a cancerous tumor or a benign tumor. One or more IONC-AAPH nanoplatforms may be positioned within a region that includes tumor tissue, is adjacent to the tumor tissue, or is within a particular threshold distance from the tumor tissue. In some embodiments, the region may be a subcutaneous region within the subject. In other embodiments, the region may include a portion of dermal tissue. In any case, after positioning the IONC-AAPH nanoplatforms within a region of the subject, a magnetic field may be applied to the region. In this way, the IONC-AAPH nanoplatform simultaneously generates local heat and free radicals within the region of the subject that induce ICD in the tumor tissue, thereby efficiently eradicating the tumor tissue in the subject.

In some embodiments, the magnetic field is an alternating magnetic field. Additionally, the magnetic field may be applied to the region via any suitable magnetic field generator. In certain embodiments, the magnetic field may be applied to the region over a first time period. Thereafter, the tumor tissue may be assessed to determine whether the tumor tissue has decreased in size. For instance, one or more quantification techniques may be performed that determine a size of the tumor tissue, a volume of the tumor tissue, a quantity of tumor cells in the tumor tissue, or the like. After determining a change in size of the tumor tissue, or lack thereof, the magnetic field may be applied to the region over a second time period based on the change in size of the tumor tissue, or lack thereof. For instance, one or more parameters associated with the IONC-AAPH nanoplatforms, one or more additional parameters associated with the magnetic field, or both, may be adjusted before applying the magnetic field to the region over the second time period. In some embodiments, the parameters associated with the IONC-AAPH nanoplatforms may include a position of the IONC-AAPH nanoplatforms with respect to the tumor tissue of the subject, a quantity of the IONC-AAPH nanoplatforms within the region of the subject, or the like.

In some embodiments, the quantification techniques may be applied to the tumor tissue before positioning the IONC-AAPH nanoplatforms within the region of the subject and/or applying the magnetic field to the region. For instance, such techniques may determine a quantitative baseline associated with the tumor tissue. The quantitative baseline (e.g., a baseline size, a baseline volume, a baseline quantity of tumor cells) associated with the tumor tissue may be compared to subsequent corresponding measurements of the tumor tissue to assess a progress of the treatment modality.

In some embodiments, a magnetotherapy treatment (e.g., applying a magnetic field to the IONC-AAPH nanoplatforms within a region of the subject) may be administered over a first time period and an ICB treatment may be administered over a second time period. The first time period may overlap with the second time period. That is, the magnetotherapy treatment may be administered to the subject over a time period that overlaps with a time period for administering the ICB treatment to the subject. For example, the magnetotherapy treatment may be administered simultaneously or substantially simultaneously with the ICB treatment. Alternatively, the first time period may not overlap with the second time period. For example, the magnetotherapy treatment may be administered sequentially with the ICB treatment.

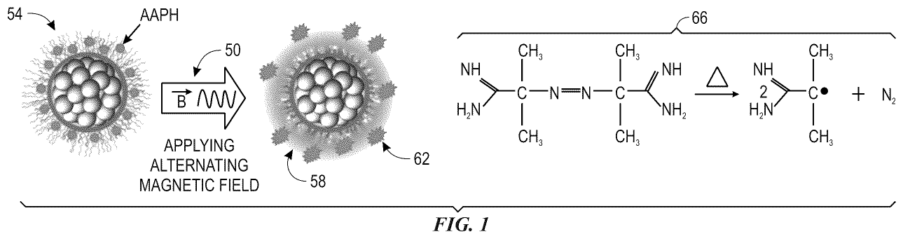

With the foregoing in mind, FIG. 1 is a schematic illustration of heat and free radical generation from a multifunctional IONC-AAPH nanoplatform under AMF. For instance, the left side of FIG. 1 illustrates a process that applies an AMF 50 to the IONC-AAPH nanoplatform 54 to simultaneously generate localized heat via magnetic hyperthermia and free radicals 62 in a region surrounding the IONC-AAPH nanoplatform 54. For instance, the IONCs act as a heating mediator of the magnetic hyperthermia. Additionally, the right side of FIG. 1 illustrates the thermal decomposition 66 of AAPH into carbon-centered free radicals.

### Synthesis and Characterization of IONC-AAPH

In certain embodiments, the IONCs may be synthesized through hydrolysis of iron salts in glycol in a solvothermal reaction. From various TEM images, the diameter of the IONCs was measured to be 40 nanometers (nm)±3.9 nm. Each nanocluster is composed of ˜300 primary magnetic iron oxide nanocrystals (MIONs) of ˜5 nm. Each primary particle is approximately 5 nm in diameter. The primary nanoparticles in each nanocluster have the same crystal orientations. The specific surface area of the nanoclusters measured by a Brunauer-Emmett-Teller (BET) surface analyzer was around half of the specific surface area of primary 5 nm MIONs, indicating that the neighboring primary nanoparticles within an IONC include at least some shared interfaces and are thus interconnected. A Raman spectrum of IONCs indicates that the primary MIONs are magnetite (Fe3O4). The IONCs were coated with poly(AA-co-AMPS-co-PEG), rendering them water-soluble and stable in cell culture media.

To produce free radicals, a water-soluble azo compound AAPH was loaded to the IONC surface through a poly(acrylic acid) (PAA) chain. In one such implementation, PAA was first reacted with a nitrodopamine molecule to form nitrodopamine-PAA. The AAPH molecules were then conjugated to nitrodopamine-PAA through reaction between the amine group on AAPH and the carboxyl group on PAA. The nitrodopamine-PAA-AAPH was attached to the IONC surface through the coordination between the catechol group on the nitrodopamine molecule and the iron atoms on the nanocluster surface. The successful loading of AAPH to the IONCs was confirmed by the spectra from Fourier transform infrared (FTIR) spectroscopy.

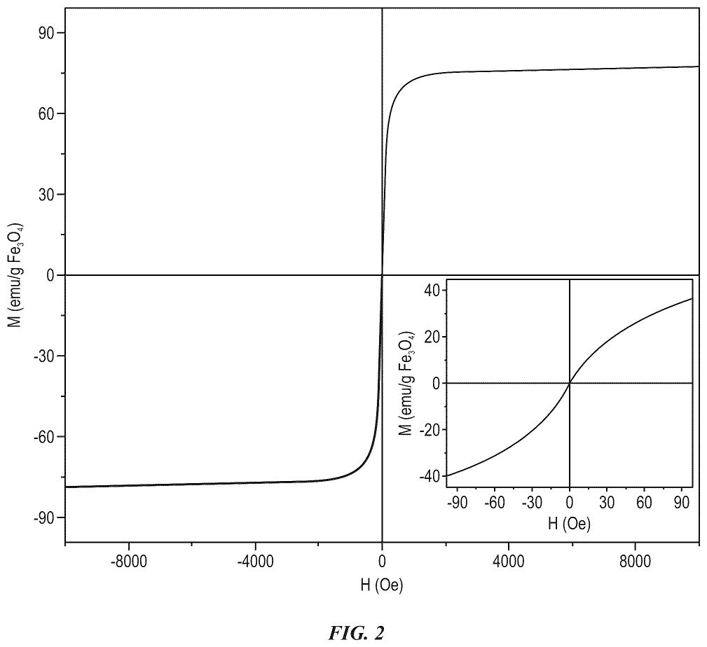

The IONC exhibits magnetic behavior of interest due to its nanostructure. FIG. 2 is a magnetization curve that shows negligible coercivity of IONC-AAPH. This confirms that the 40 nm IONCs remain superparamagnetic. The IONC can generate heat quickly under an alternating magnetic field (AMF), leading to an increase of the solution temperature. By way of example, in one implementation the IONCs (at 0.5 mg Fe/mL concentration) can generate heat rapidly under AMF of 9.35 kA/m and 320 kHz, resulting in a temperature increase of 25° C. in water within 200 s. The specific absorption rate (SAR) of the 40 nm IONCs in this example was 564±27 W/g Fe, which was 40-fold higher than that of the primary MIONs.

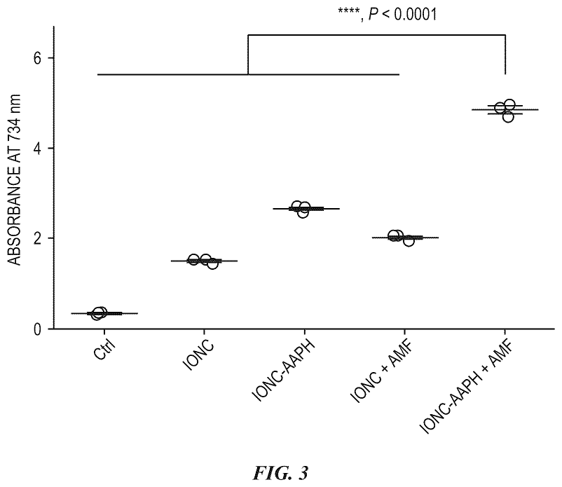

As discussed herein, the heat from IONCs under AMF can accelerate the generation of free radicals from the decomposition of AAPH. For example, the ability to generate free radicals by IONC-AAPH was measured using 2,2′-Azobis(2-methylpropionamidine) dihydrochloride (ABTS), which acts as a free radical indicator. ABTS reacts with free radicals and forms ABTS+·, which exhibits a characteristic absorbance spectrum between 400 nm and 950 nm, with a peak absorbance spectrum at 734 nm. FIG. 3 illustrates the quantification of ABTS+· absorbance under various conditions. In particular, PBS containing IONC or IONC-AAPH of 300 μg Fe/mL concentration was mixed with ABTS solution and then incubated at 37° C. or subjected to AMF for 1 h, followed by removal of the nanoclusters from the samples using centrifugal filters. The generation of ABTS+· was then determined by measuring the absorbance spectra of the samples. As shown in FIG. 3, for the sample with IONC-AAPH under AMF, the absorbance peak at 734 nm due to ABTS+· was significantly higher than that of other groups (PBS as control, IONC, IONC-AAPH without AMF, and IONC under AMF), indicating free radical generation due to decomposition of AAPH.

### Intracellular Free Radical Generation

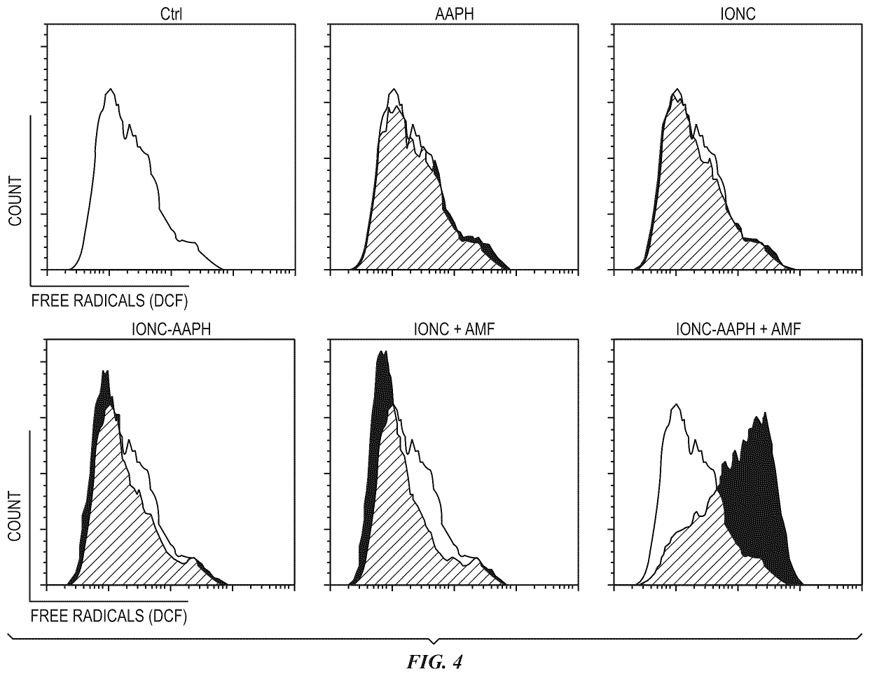

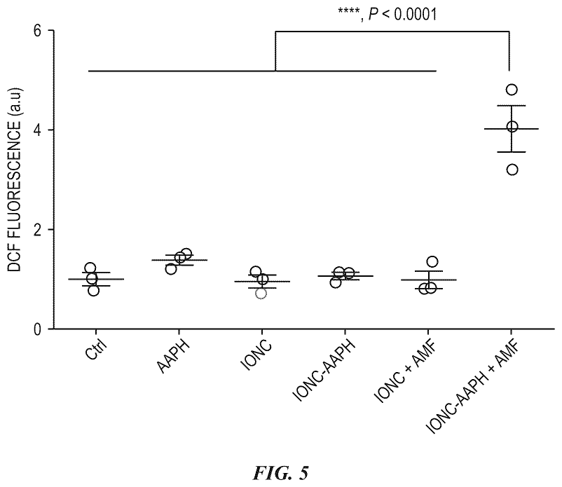

Additionally, intracellular free radical generation by IONC-AAPH was measured using 2′,7′-dichlorodihydrofluorescein diacetate (H₂DCFDA) as an intracellular free radical probe. H₂DCFDA is a non-fluorescent cell-permeant compound. Upon oxidation by free radicals, H₂DCFDA is converted to a highly fluorescent 2′,7′-dichlorofluorescein (DCF). MC-38 cells (murine colon adenocarcinoma cell line) were pre-incubated with IONC or IONC-AAPH (at 300 μg Fe/mL concentration) for 1 h to allow internalization into cells. The intracellular IONC and IONC-AAPH were 0.79±0.16 and 0.87±0.12 pg Fe/cell, respectively, as quantified by a ferrozine-based colorimetric assay. After internalization, the cells were exposed to AMF for 1 h. The intracellular free radicals in the MC-38 cells were then examined with H2DCFDA through fluorescence microscopy and flow cytometry. FIG. 4 illustrates a flow cytometry analysis of intracellular free radicals in MC-38 cells detected via fluorescence from DCF. Further, FIG. 5 is a graph illustrating the quantification of intracellular free radicals (e.g., quantification of the mean fluorescence of DCF). The data in FIG. 5 represents the mean DCF fluorescence of each group +the standard error of mean (s.e.m.). As shown by FIGS. 4-5, there was negligible fluorescence in control cells and cells treated with free AAPH, IONC, IONC-AAPH, or magnetic hyperthermia (IONC+AMF). In contrast, as shown by FIGS. 4-5, the cells treated with IONC-AAPH under AMF showed fluorescence as detected by both fluorescence microscopy and flow cytometry. This indicates that IONC-AAPH can generate a high level of free radicals under AMF actuation.

### In vitro Tumor Cell Killing

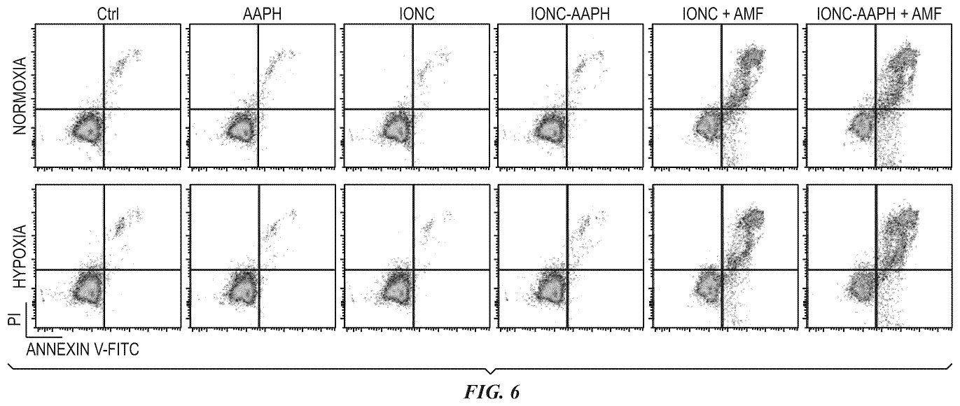

Further, the cell-killing effect of IONC-AAPH under AMF actuation in vitro was also examined through fluorescence microscopy and flow cytometry. For example, FIG. 6 illustrates a flow cytometry analysis of cell death in MC-38 cells after different treatment under normoxic and hypoxic conditions. To determine the ability of IONC-AAPH to kill tumor cells, MC-38 cells were treated with IONC-AAPH and subjected to AMF for 2 h under normal level of oxygen (normoxia) and the cell death was imaged fluorescently by co-staining cells with calcein AM (for live cells) and propidium iodide (PI, for dead cells). Cell death was further quantified by flow cytometry analysis of Annexin-V and PI double-stained cells (FIG. 6). It was found that under AMF, IONC-AAPH caused a higher level of cell death compared with IONC only (52.1% vs 38.1% as quantified by flow cytometry as shown in FIG. 6). Without AMF, IONC-AAPH did not kill MC-38 cells, indicating a good biocompatibility.

Given that the generation of free radicals from AAPH is independent of oxygen, the cell killing ability of IONC-AAPH under hypoxic condition was also evaluated by pretreating MC-38 cells with 100 micromole per liter (μmol/L) cobalt chloride (CoCl₂) for 24 h. Hypoxia was successfully induced as evidenced by the upregulation and accumulation of hypoxia-inducible factor 1-α (HIF-1α). As shown in FIG. 6, under hypoxia, IONC-AAPH maintained a higher cell killing ability than IONC alone (53.3% vs 34.3% as quantified by flow cytometry as illustrated in FIG. 6). These results indicate that magnetic heating and the carbon-centered free radical from AAPH can kill tumor cells effectively and synergistically, even under hypoxic conditions. Since carbon-centered free radicals from AAPH can be indued by heat independent of oxygen, the approach using IONC-AAPH has a clear advantage over therapeutic strategies based on ROS generation by radiation or light, which is often hindered by the low oxygen levels in the TME.

### Mechanisms of IONC-AAPH-mediated Cell Killing

Hyperthermia treatment may induce lipid peroxidation, DNA damage, protein denaturation, and cell organelle disruption. In addition, carbon-centered free radicals generated from AAPH are highly reactive and can damage lipid, DNA, protein, and other biomolecules, leading to cell death. Thus, to understand the cell-killing mechanisms of IONC-AAPH under AMF, the effects of IONC-AAPH on various cellular components, including DNA, F-actin, mitochondria, gluthathione (GSH), and lipids, were evaluated. γ-H2AX is a sensitive marker of DNA damage and has been extensively used in research and clinical studies. It forms foci around the site of DNA double-strand break (DSB). Based upon a γ-H2AX foci analysis of DNA damage, it was observed that hyperthermia treatment (IONC+AMF) induced obvious γ-H2AX foci formation, indicating the DNA damaging activity of IONC-mediated hyperthermia. When combined with free radical generation (i.e., treating cells with IONC-AAPH under AMF for 90 min), the foci number and fluorescence intensity were dramatically increased.

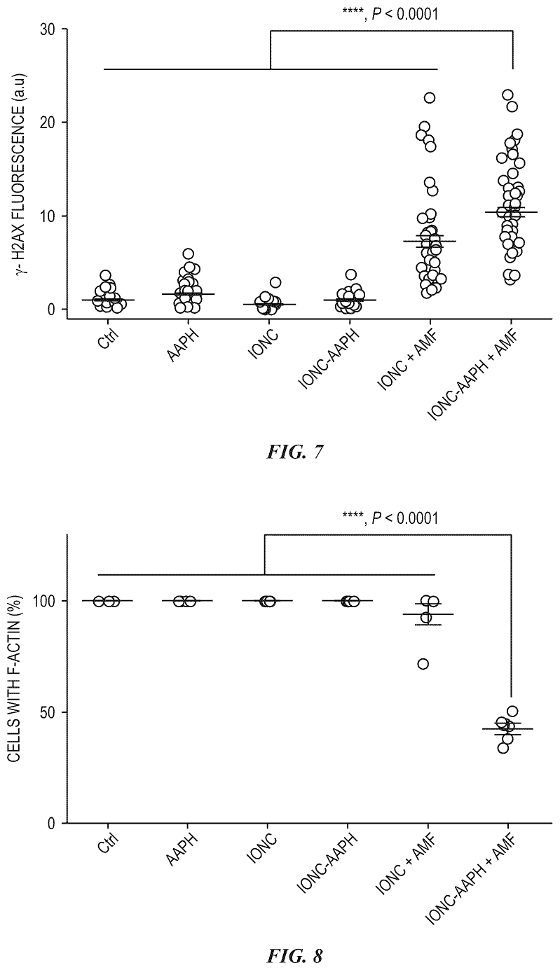

FIG. 7 depicts the quantification of γ-H2AX expression based on fluorescence intensity. As shown in FIG. 7, the combined heat and free radical generation by IONC-AAPH caused higher DNA damage compared with hyperthermia alone. The low numbers of γ-H2AX foci observed in the four control groups (cells only, AAPH, IONC, and IONC-AAPH without AMF) may be due to the spontaneous DNA damage in the MC-38 cells, as spontaneous γ-H2AX foci have also been found in several cancer cell lines and some cancer tissues.

To examine the effect of heating and free radicals on the actin cytoskeleton, actin filaments (F-actin) were stained with fluorescent phalloidin. Without heating, F-actin in cells treated with AAPH, IONC, and IONC-AAPH was well organized and elongated, similar to cells-only as control. The MC-38 cells treated with IONC and subjected to AMF (magnetic hyperthermia) rounded up and showed retracted actin filaments. In contrast, for cells treated with IONC-AAPH and subjected to AMF, more than 50% lost the F-actin structure (i.e., no phalloidin staining), suggesting that the actin filaments in these cells were disrupted by the combination of heat with free radicals. In cells subjected to heating and free radicals but still showing phalloidin staining, the actin filaments were further retracted and more condensed compared with cells under heating only. FIG. 8 illustrates a quantification of MC-38 cells with F-actin structure in the above-described morphological analysis. As shown, the cells treated with magnetic hyperthermia (IONC+AMF) rounded up and showed retracted actin filaments. In contrast, in the magnetotherapy group (IONC-AAPH+AMF), more than 50% of the MC-38 cells lost F-actin structure, indicating that the actin filaments in the MC-38 cells were completely disrupted by the combination of heat with free radicals. In the cells with phalloidin staining, the actin filaments were further retracted and more condensed compared with the hyperthermia only group. This suggests the actin cytoskeleton disruption capability of IONC-AAPH-mediated magnetotherapy.

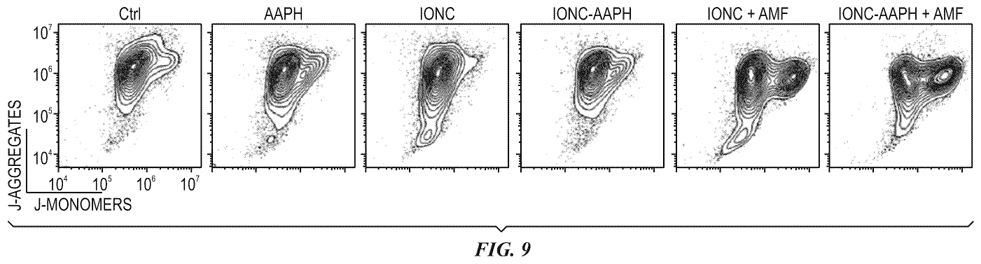

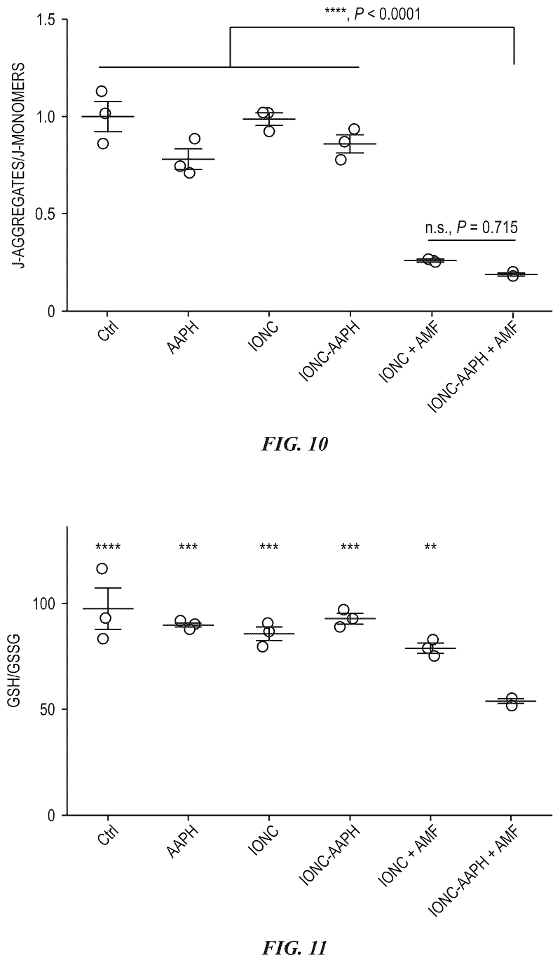

JC-1 dye was used to examine the effect of heat and free radical generation by IONC-AAPH on mitochondria. JC-1 dye is an indicator of mitochondrial membrane potential (MMP). In healthy cells with high MMP, JC-1 accumulates in the mitochondria and forms J-aggregates, which emit red fluorescence. In contrast, apoptotic cells with low MMP, JC-1 diffuses in the cytoplasm and remains as J-monomers, which give green fluorescence. A decrease in J-aggregates to J-monomers ratio indicates MMP loss (i.e., mitochondrial depolarizations). J-aggregates were formed in the control cells and cells treated with AAPH, IONC, and IONC-AAPH (without heating or free radicals) as determined from fluorescent images. In contrast, there were only J-monomers in the cells treated with IONC or IONC-AAPH under AMF, suggesting that depolarization of mitochondria occurred due to heat or the combination of heat and free radicals. The J-aggregates to J-monomers ratios quantified using flow cytometry confirmed the significant MMP loss due to heat and free radicals, as illustrated in FIGS. 9 (depicting results of flow cytometry analysis of mitochondrial membrane potential of MC-38 cells 4 h after treatment by JC-1 staining) and 10 (depicting quantification of the J-aggregate to J-monomer ratio using the mean fluorescence detected by flow cytometry in FIG. 9). No significant difference was observed between having heat only and having heat plus free radicals (FIG. 10), suggesting that it was mainly magnetic heating that caused mitochondrial depolarization in MC-38 cells.

Glutathione (GSH) is an antioxidant that plays an important role in the scavenging of intracellular free radicals and protecting the cells against oxidative damage. GSH depletion will disrupt the redox homeostasis and lead to oxidative stress and eventual cell death. MC-38 cells were treated with 300 μg Fe/mL of IONC or IONC-AAPH and subjected to AMF for 2 hours. The intracellular GSH was quantified after 2 hours of incubation at 37° C. Intracellular GSH was also measured for cells treated with AAPH, IONC, or IONC-AAPH, respectively, without AMF and incubated at 37° C. for 4 h. The intracellular GSH was quantified using a luminescence-based GSH-Glo glutathione assay. FIG. 11 illustrates a GSH depletion measurement of the MC-38 cells 2 hours after treatment. FIG. 11 shows that treating MC-38 cells with free AAPH, IONC, and IONC-AAPH, respectively, did not affect the GSH to GSSG ratio, and magnetic hyperthermia (IONC with AMF) only caused a slight decrease in GSH/GSSG. In contrast, treating cells with IONC-AAPH under AMF (i.e., with both heating and free radicals) decreased the GSH/GSSG ratio to 55% of that in the control cells. These results suggest that IONC-AAPH under AMF can cause significant GSH depletion and thus an increased level of oxidative stress.

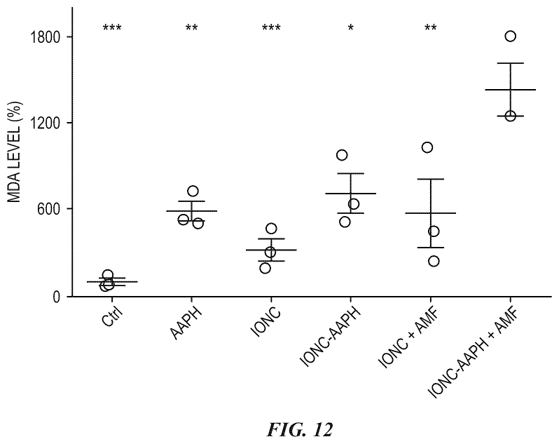

Further, the effect of IONC-AAPH induced heat and free radicals on lipids was quantified by measuring the level of malondialdehyde (MDA). Malondialdehyde (MDA) is one of the final products of lipid peroxidation. MDA is a widely used biomarker of oxidative stress. Accordingly, the extent of lipid peroxidation after IONC-AAPH treatment was measured via MDA levels. FIG. 12 illustrates a quantification of MDA levels in MC-38 cells 2 hours after treatment. It was observed that the MC-38 cells treated with free AAPH, IONC, and IONC-AAPH, respectively, without AMF, as well as cells treated with IONC under AMF had some increase in MDA levels compared with that of control cells, as shown in FIG. 12. The low levels of lipid peroxidation in these groups may have resulted from the free radicals generated from the slow decomposition of AAPH at 37° C., or the IONC induced fenton/fenton-like reaction, or their combination (IONC-AAPH without AMF). Cells treated with IONC-AAPH under AMF induced a significant increase in the MDA level. These results indicate that heat and free radicals generated by IONC-AAPH induced lipid peroxidation, which in turn caused cancer cell death.

Taken together, the above results demonstrate that IONC-AAPH under AMF can cause damage to multiple cellular components, including DNA, actin cytoskeleton, mitochondria, and lipid membranes, as well as significant intracellular GSH depletion. All of these may contribute to the cancer cell death triggered by IONC-AAPH.

### Immunogenic Cell Death and Dendritic Cell Maturation Triggered By IONC-AAPH-under AMF

Immunogenic cell death (ICD) is characterized by the exposure or release of damage-associated molecular patterns (DAMPs), such as calreticulin (CRT), heat shock protein 70 (HSP70), and adenosine triphosphate (ATP). To determine if IONC-AAPH under AMF induced immunogenic cell death, the cell surface translocation of CRT (ecto-CRT) and Hsp70 (ecto-Hsp70) and the release of ATP to extracellular space were examined at different time points after treating MC-38 cells with IONC or IONC-AAPH under AMF for 2 h or with AAPH, IONC, or IONC-AAPH without AMF (defined as “treatment” here).

The results showed that the translocation of CRT and Hsp70 to the cell surface happened within 2 h after treatment with IONC and IONC-AAPH under AMF. At 6 h post-treatment, the ecto-CRT and ecto-Hsp70 levels of MC-38 cells treated with heating and free radicals were still significantly higher than those of other groups (cells only, AAPH, IONC and IONC-AAPH without AMF, and IONC with AMF). Additionally, 24 h after treatment with IONC-AAPH, the expression of Hsp70 protein increased by 27-fold compared with control. It was further found that there was a high level of extracellular ATP due to heat and free radicals. At 2 h post-treatment with IONC-AAPH, the concentration of extracellular ATP was 130.4 nM, ˜420-fold higher than that of cells only (control). At 6 h post-treatment with IONC-AAPH, the level of extracellular ATP was still ˜100-fold higher than the control. Heating only without free radicals also showed significant increase in extracellular ATP, ˜210-fold and ˜11-fold higher than control, respectively, at 2 and 6 h post-treatment.

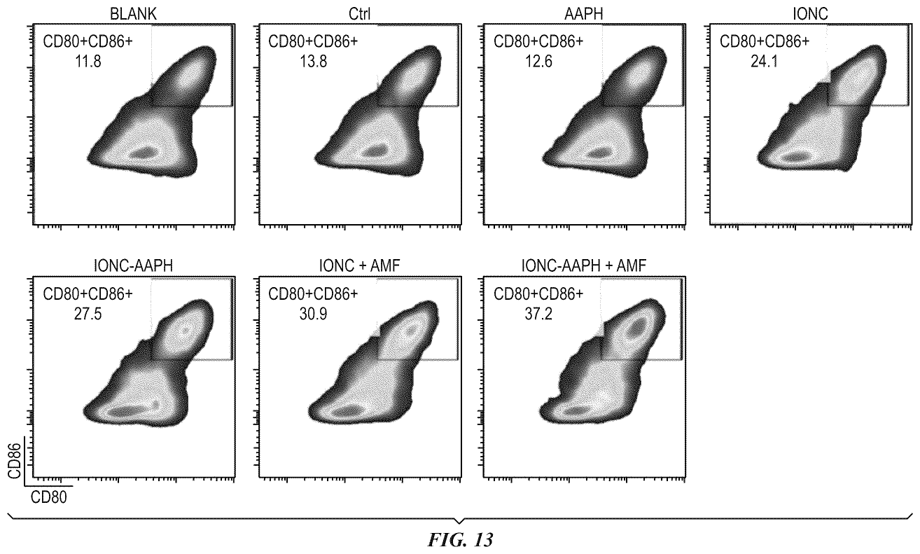

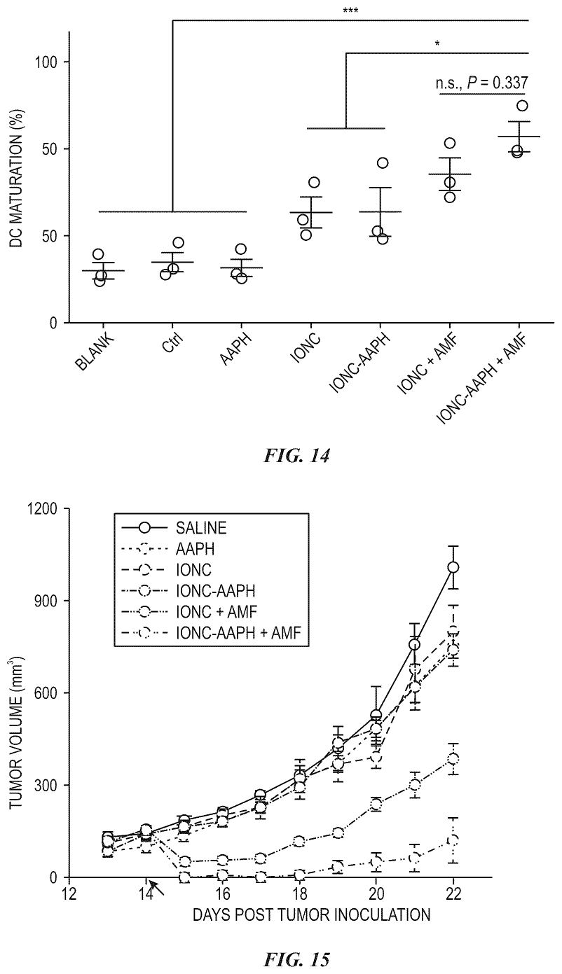

These results suggest that the cell death due to IONC-AAPH under AMF is highly immunogenic. Dendritic cells (DCs) are a key type of antigen-presenting cells that play critical roles in initiating and regulating antitumor immune response. To determine if the immunogenic cell death induced by magnetotherapy can activate dendritic cells, bone marrow-derived dendritic cells (BMDCs) were cocultured with IONC-AAPH-treated MC-38 cells, together with the medium containing DAMPs, in a transwell system for 24 h. The maturation of dendritic cells was estimated as the percentage of CD80+CD86+ cells in CD11c+ cells. As shown in FIGS. 13 (depicting the quantification of DC maturation using representative flow cytometry data) & 14 (depicting the quantification data of FIG. 13), cells alone or the cells treated by AAPH did not show a noticeable effect on DC maturation. The percentage of mature DCs in IONC and IONC-AAPH groups was 2.1-fold of the blank. Given that incubation with IONC and IONC-AAPH had little effect on MC-38 cells, the increase of DC maturation with these two groups without AMF was likely induced by the internalized nanoclusters The DC maturation in magnetic hyperthermia group (IONC+AMF) and magnetotherapy group (IONC-AAPH+AMF) was 2.8- and 3.6-fold of the blank, respectively. The significantly increased DC maturation in the magnetotherapy group indicates that the immunogenic cell death caused by IONC-AAPH under AMF can effectively activate DCs.

### Anti-tumor Activity of IONC-AAPH under AMF

The antitumor activity of IONC-AAPH in vivo was evaluated with a subcutaneous MC-38 mouse tumor model. The subcutaneous tumor was induced by inoculating MC-38 cells into the right flank of respective C57BL/6N mice. The mice were randomly divided into 6 groups (n=3): (i) saline, (ii) AAPH, (iii) IONC, (iv) IONC-AAPH, (v) IONC+AMF, and (vi) IONC-AAPH+AMF. When the tumor volume reached 150-200 mm³, the mice were intratumorally injected with saline, AAPH (2 mM), IONC (5 mg Fe/mL), and IONC-AAPH (5 mg Fe/mL), respectively. After injection the mice in IONC+AMF and IONC-AAPH+AMF groups were subjected to AMF for 1 h. The temperature of the tumors injected with IONC or IONC-AAPH increased rapidly and reached above 45° C. within 5 min, as measured by an infrared thermal camera.

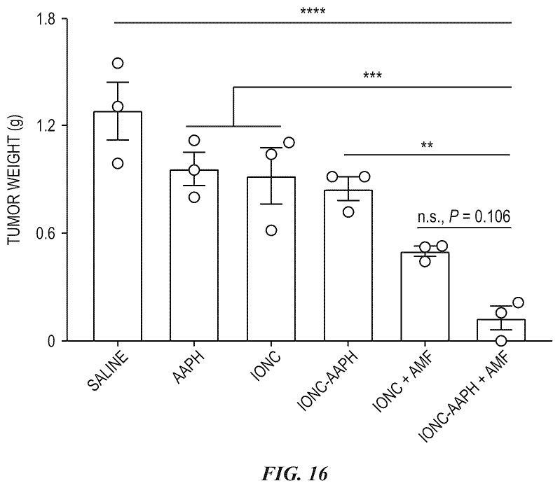

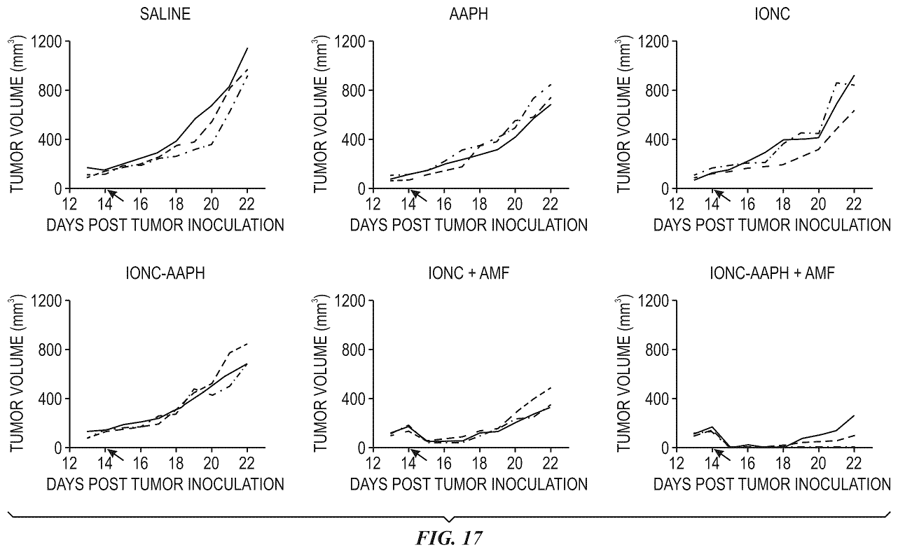

After applying AMF, the temperature in the tumor was much higher than in other parts of the body. In contrast, a negligible temperature increase was observed in the tumors injected with saline and treated with AMF. As shown in FIG. 15 (depicting the tumor growth curves of different treatment groups, with treatment onset at day 14) and FIG. 17 (depicting the tumor growth curves of the individual mice, with treatment onset at day 14), IONC-induced hyperthermia (IONC+AMF) reduced the tumor burden effectively and delayed the regrowth of tumor from the residual tumor cells. When combining local heating with free radical generation by IONC-AAPH under AMF, tumor growth was significantly inhibited more than with hyperthermia alone. Specifically, at the end point (Day 22), the average tumor volume in mice treated with IONC-AAPH under AMF was reduced by 88.2% compared with control tumors (injected with saline), whereas with IONC alone under AMF the average reduction in tumor volume was only 61.7%. The reduction of tumor weight due to heat and free radicals generated by IONC-AAPH was higher than hyperthermia alone, while injecting AAPH, IONC, and IONC-AAPH without applying AMF only slightly reduced the tumor weight, as shown in FIG. 16 (depicting the weights of excised tumors as means ±s.e.m). The synergy between heat from IONC under AMF and free radicals generated from AAPH is likely due to free radicals sensitizing the tumor cells to heat thus enhancing heat-induced cell killing.

The hematoxylin and eosin (H&E) staining and terminal deoxynucleotidyl transferase dUTP nick end labeling (TUNEL) analysis of the tumor tissues demonstrated that injecting IONC-AAPH and applying AMF caused significant tumor tissue damage, higher than hyperthermia alone; no tumor tissue damage was found in the control groups (injecting saline, AAPH, IONC, IONC-AAPH without AMF). In addition, the analysis of Ki-67 expression in tumor tissues indicates that tumor cell proliferation was inhibited efficiently by IONC-AAPH under AMF. Taken together, these results suggest that the combination of local heat and free radicals by IONC-AAPH led to a profound antitumor effect in vivo.

### Abscopal Effect of Magneto-Immunotherapy (Mag-IT)

Evaluation was performed of whether magneto-immunotherapy that combines IONC-AAPH-based local heat and free radical generation with anti-PD-1 immune checkpoint blockade (ICB) therapy can induce an abscopal antitumor effect. Several local cancer treatment modalities, such as radiation therapy and phototherapy, have been reported to induce an abscopal effect on the untreated distant tumors. Previous studies have shown that the abscopal effect is attributed to the systemic antitumor immune responses triggered by immunogenic cell death during local treatment and combining local treatment strategies with immunotherapy can amplify the abscopal effect.

In vitro studies as described herein have demonstrated the ability of IONC-AAPH to induce immunogenic cell death. To determine the abscopal effect, a biliteral subcutaneous tumor model was used by inoculating MC-38 cells into the right and left flanks of the same mouse. The tumor on the right side was designated as the primary tumor, subjected to different treatment, and the tumor on the left side was designated as secondary (distant) tumor without any treatment. The mice were randomly divided into 6 groups (n=8): (i) saline, (ii) anti-PD-1, (iii) IONC-AAPH, (iv) IONC-AAPH+anti-PD-1, (v) IONC-AAPH+AMF, and (vi) IONC-AAPH+AMF+anti-PD-1. When the volume of the primary tumor reached 50-100 mm³, saline or IONC-AAPH was injected directly into the primary tumor. Mice in groups (v) and (vi) were treated with IONC-AAPH under AMF for 1 h. For mice in groups (ii), (iv), and (vi), anti-PD-1 antibody was injected intraperitoneally at 10 mg/kg of body weight on Days 8, 11, and 14. The tumor growth was monitored daily.

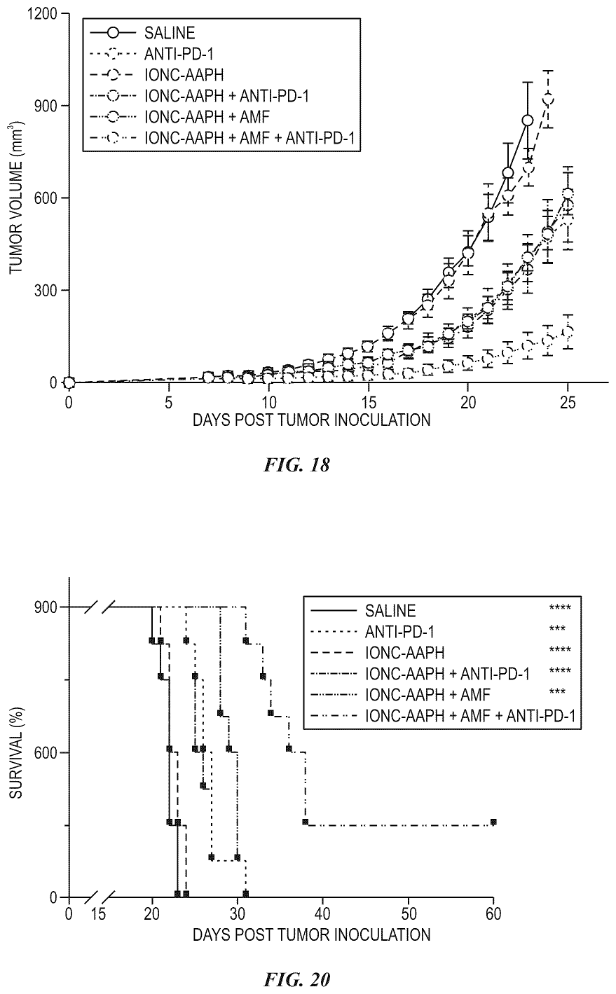

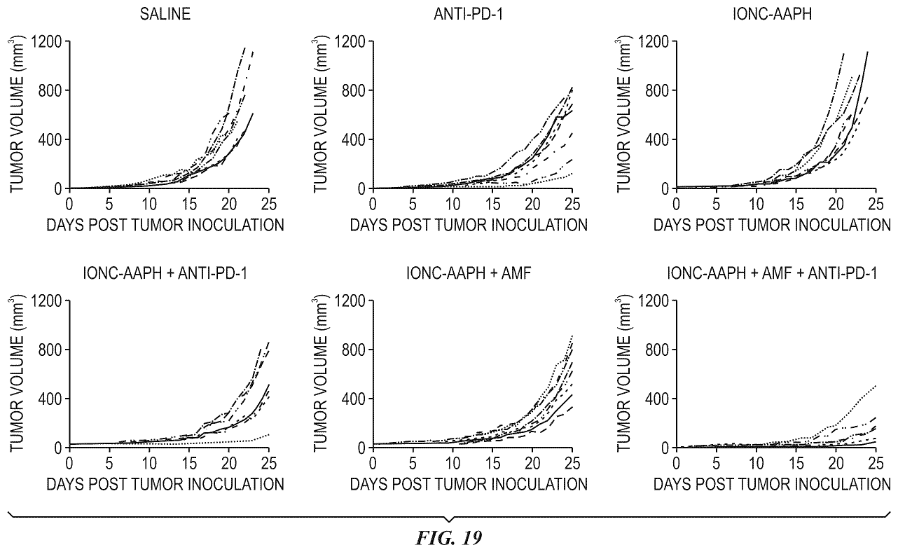

Injecting IONC-AAPH into the primary tumor without AMF showed no inhibitory effect on the secondary tumor, as illustrated in FIG. 18 (depicting the average growth curve of the secondary tumor) and FIG. 19 (depicting the tumor growth curves of individual mice corresponding to FIG. 18). In contrast, as shown in FIG. 18 and FIG. 19, injecting IONC-AAPH into the primary tumor and subjecting the mice to AMF partially inhibited the growth of the secondary tumor, suggesting a moderate abscopal effect due to local heat and free radical generation. When combining heat and free radical produced by IONC-AAPH with anti-PD-1 ICB, the abscopal effect on the secondary tumor was dramatically enhanced, as demonstrated by the much slower growth of the secondary tumor compared with the saline control group. Injection of anti-PD-1 antibody also partially delayed the growth of the secondary tumor due to the activation of systemic immune response. However, all the animals injected with anti-PD-1 antibodies reached the end point within 31 days, as shown in FIG. 20 (depicting the survival curves of mice receiving different treatments (n=8)). In comparison, the combination of local heat, free radicals, and anti-PD-1 ICB eradicated both the primary and secondary tumors in 25% of the treated mice, which survived throughout the study period (60 days) with no tumor relapse, as shown in FIG. 20. Together, these findings suggest that magneto-immunotherapy elicits a strong abscopal effect to suppress the growth of distant tumors and has the potential to treat metastatic cancers.

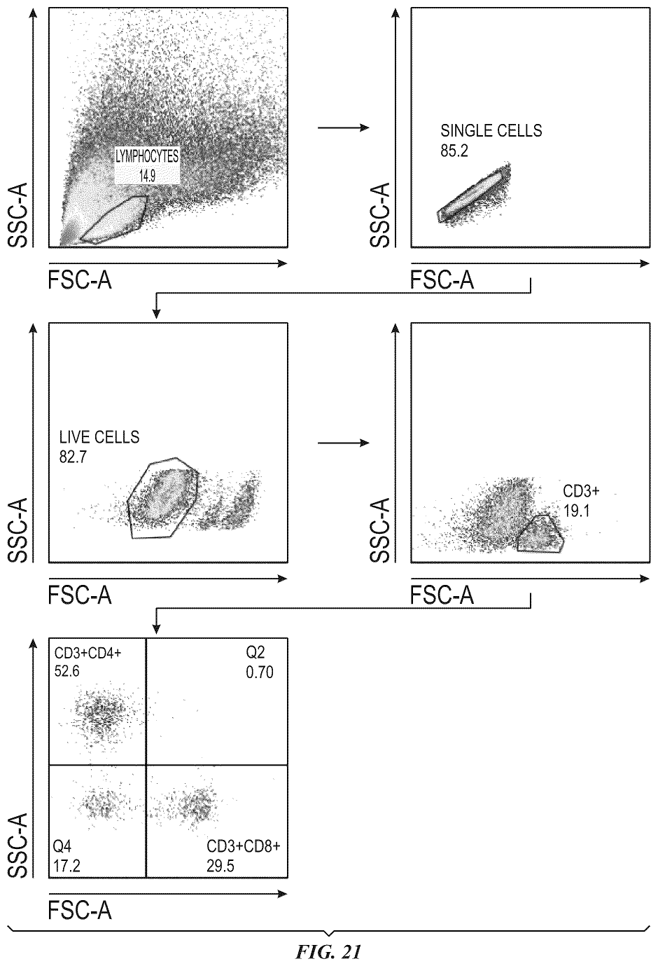

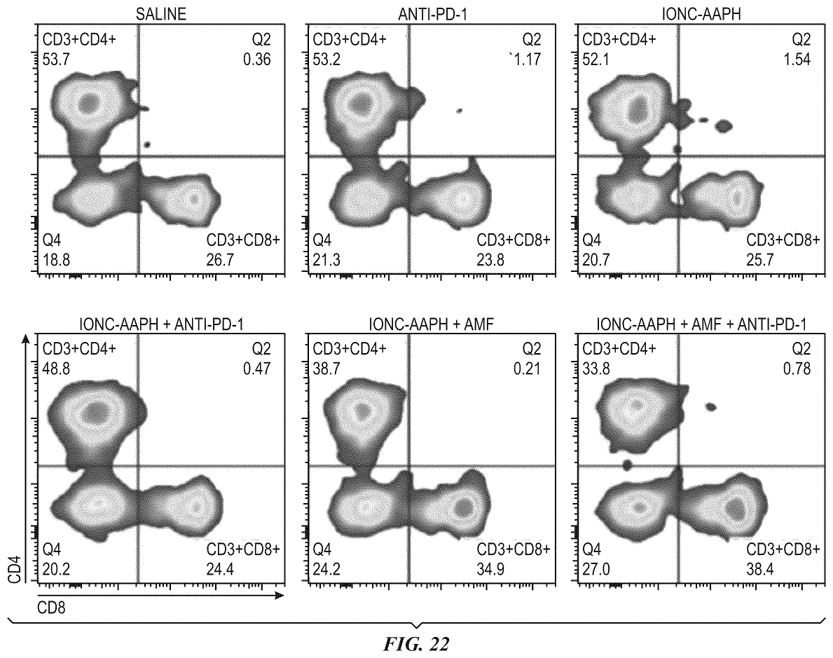

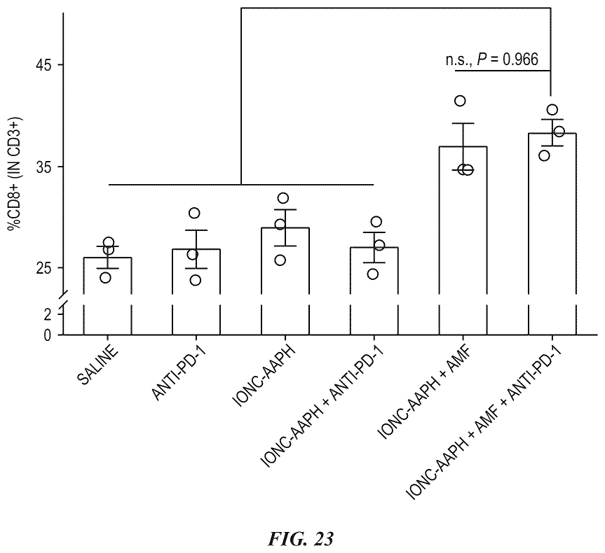

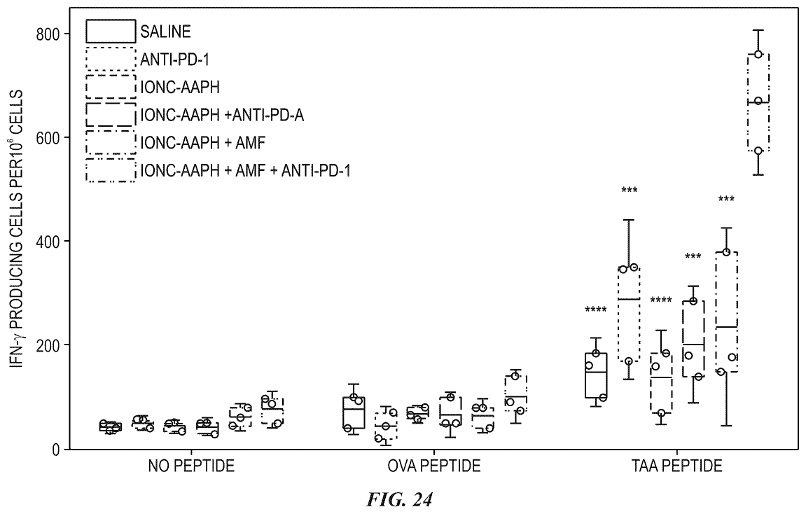

The mechanisms underlying the observed abscopal effect were further investigated. The tumor infiltrating lymphocyte profiles in the untreated tumors were analyzed. Mice with bilateral MC-38 tumors were treated as described in the preceding discussion. On Day 17 (i.e., 10 days after treating the primary tumor), the secondary tumors without any treatment were harvested for flow cytometry analysis. The gating strategies are visually illustrated in FIG. 21, which depicts the representative gating strategy for analyzing tumor infiltrating lymphocytes in secondary tumors. FIGS. 22 and 23 depict infiltrating immune cells in the secondary tumors (n=3), with FIG. 22 illustrating representative flow cytometry data and FIG. 23 illustrating the corresponding quantification data. As shown in FIGS. 22 and 23, the percentage of CD3⁺CD8⁺ T cells in the secondary tumors increased in the mice receiving magnetotherapy (IONC-AAPH with AMF) or magneto-immunotherapy (IONC-AAPH with AMF plus ICB). There was no significant difference between these two groups. These results suggest that treating the primary tumor with magnetotherapy can increase the number of CD3⁺CD8⁺ T cells in the untreated secondary tumor. The tumor-specific immune response post magneto-immunotherapy was also evaluated by enzyme-linked immunospot (ELISpot) assay. The splenocytes were harvested on Day 17 and stimulated for 24 h with KSPWFTTL, a tumor-associated antigen (TAA) peptide. Splenocytes incubated with ovalbumin (OVA) peptide SINFEKL or without peptide were included as control groups. FIG. 24, depicts quantification data in which tumor antigen-specific T cells determined by ELISpot assay (n=3) and where IFN-γ secreting T cells were detected 24 h after stimulation and in which splenocytes without peptide stimulation were included as control groups (data represent mean ±s.e.m.). As shown in FIG. 24, the number of antigen-specific IFN-γ producing T cells was significantly higher in the mice treated with magneto-immunotherapy (668±93 per 10⁶ cells), which was 4.5- and 2.8-fold higher than that of the saline control (148±44 per 10⁶ cells) and magnetotherapy (235±126 per 10⁶ cells) groups, respectively. These results indicate that magneto-immunotherapy of the primary tumor can efficiently activate systemic tumor-specific T-cell response. Thus, the strong abscopal effect of magneto-immunotherapy may result from the combination of increased tumor infiltrating CD8⁺ T cells and enhanced tumor-specific T-cell response.

### Long-term Immune Memory Effect of Magneto-Immunotherapy (Mag-IT)

Immune memory is the hallmark of the adaptive immune response that is essential for long-term protection against pathogens including tumor cells. Upon a second encounter with the same tumor cells, memory T cells can rapidly respond and mount a much stronger and more effective immune response than the first immune response. IONC-AAPH under AMF was therefore investigated to see if it would induce immune memory against tumor rechallenge. The first tumor was inoculated by subcutaneous injection of MC-38 cells into the right flank of the C57BL/6N mouse. When the tumor volume reached 50-100 mm³, the tumors were removed by 1-2 rounds of treatment. The mice treated with magneto-immunotherapy (IONC-AAPH with AMF and ICB) were intraperitoneally injected with anti-PD-1 antibody (10 mg/kg of body weight) 1, 4, and 7 days after the first round of magnetotherapy. After the first tumors were completely eradicated, the mice were housed for an additional 40 days to allow the possible establishment of immune memory. The mice were then rechallenged with MC-38 cells on the contralateral side. Three naive mice (without previous tumor implant) were inoculated with MC-38 cells and used as a control. It was observed that the tumors in the naive mice grew rapidly and reached the end point within 24 days, while the growth of the second tumors in the mice treated with IONC-AAPH under AMF were completely inhibited or significantly delayed. Two out of three mice rejected the tumor rechallenge. All three of the mice cured by magneto-immunotherapy (IONC-AAPH+AMF+anti-PD-1) completely rejected the tumor rechallenge and survived throughout the study period. No tumor relapse occurred over a year post the magneto-immunotherapy. These findings are believed to demonstrate that IONC-AAPH under AMF induced long-term protective immune memory against the tumors.

### Biocompatibility Evaluation of IONC-AAPH

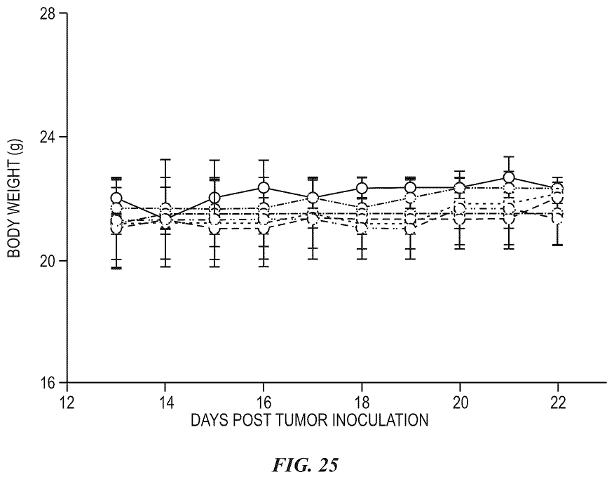

To evaluate the biocompatibility and/or potential toxicity of IONC-AAPH, the in vivo biodistribution of IONC-AAPH was examined using Prussian blue iron staining. The IONC-AAPH was directly injected into the tumor, and the tumor was completely eradicated by IONC-AAPH under AMF. The iron staining was only found in the tumor-draining lymph node (TDLN) and the spleen, which might be due to transportation within the lymphatic system. No IONC was found in the contralateral inguinal lymph node and other vital organs. The mouse body weight was also monitored, the morphology of vital organs examined, and the indices of liver and kidney functions evaluated. It was found that there were no large fluctuations of body weight in IONC-AAPH-treated mice, as shown in FIG. 25 (depicting the body weights of the mice under different assay conditions and recorded daily). The mice behaved normally after the treatment. The results of histological examination showed that there was no morphological change or apparent injury in the vital organs. Compared with the control mice, there was no significant difference in the blood levels of alanine transaminase (ALT), aspartate aminotransferase (AST), alkaline phosphatase (ALP), blood urea nitrogen (BUN), and creatinine in IONC-AAPH treated mice, suggesting that there was no adverse effect on the liver and kidney functions. Together, these findings indicate that IONC-AAPH is biocompatible and the magneto-immunotherapy using IONC-AAPH is safe.

### Alternative Implementations and Additional Study Results

While the preceding discussion relates to the efficacy of cluster-based magnetic heating in killing MC-38 cells. The following examples and discussion further illustrate that multiple other cancer cell lines, including B16F10 (murine melanoma) as well as mT4 and mT5 (murine pancreatic cancer cell lines), can also be killed using magnetic heating.

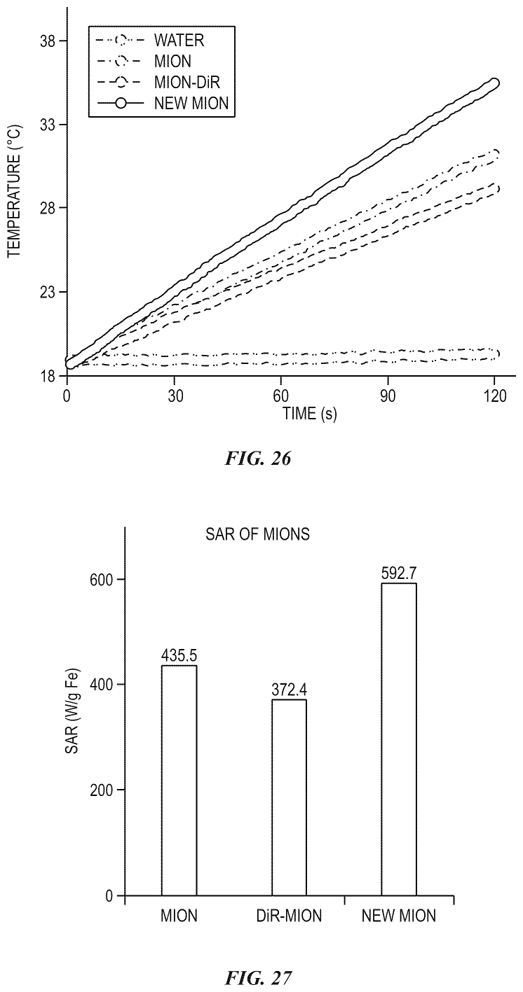

In addition to the previously described clusters, and as described herein, isolated magnetic iron oxide nanoparticles (MIONs) were employed. By way of example, in one embodiment MIONs having a diameter of approximately 19 nm were evaluated. Turning to FIG. 26, the respective heating curves of three types of MIONS, including the 19 nm MIONs, and water is illustrated. Similarly, the heating efficiency (characterized as the specific absorption rate (SAR) of these MIONs is illustrated graphically in FIG. 27.

In one such study utilizing the 19 nm MIONs, 100 μg Fe/ml of MIONs under AMF were demonstrated to kill B16F10 via MION-mediated heating in a concentration-dependent manner. In particular, the B16F10 cells were treated with 100, 200, or 300 μg Fe/mL of MIONs with or without AMF for 2 h. After treatment, the cells were stained with calcein AM and Propidium Iodide (PI) to label live and dead cells, respectively. Without AMF, no cells were killed by MIONs. Under AMF, approximately 100% of cells were killed by MION-based magnetic heating.

In a further study utilizing the 19 nm MIONs, it was shown that murine pancreatic cancer cell line mT4 can be killed by MIONs under AMF in a concentration-dependent manner. The mT4 cells were treated with 100, 200, or 300 μg Fe/mL of MIONs with or without AMF for 2 h. After treatment, the cells were stained with calcein AM and Propidium Iodide (PI) to label live and dead cells, respectively. Without AMF, no cells were killed by MIONs. Under AMF, approximately 100% of cells were killed by MION-based magnetic heating.

In another study utilizing the 19 nm MIONs, it was shown that murine pancreatic cancer cell line mT5 can be killed by MIONs under AMF in a concentration-dependent manner. In this example, the mT5 cells were treated with 100, 200, or 300 μg Fe/mL of MIONs with or without AMF for 2 h. After treatment, the cells were stained with Annexin V-FITC and Propidium Iodide (PI) to label dead cells. Live cells are negative for both Annexin V-FITC and PI. The percentage of dead cells were calculated as:

% of dead cells=100%−% of cells negative for both Annexin V-FITC and PI

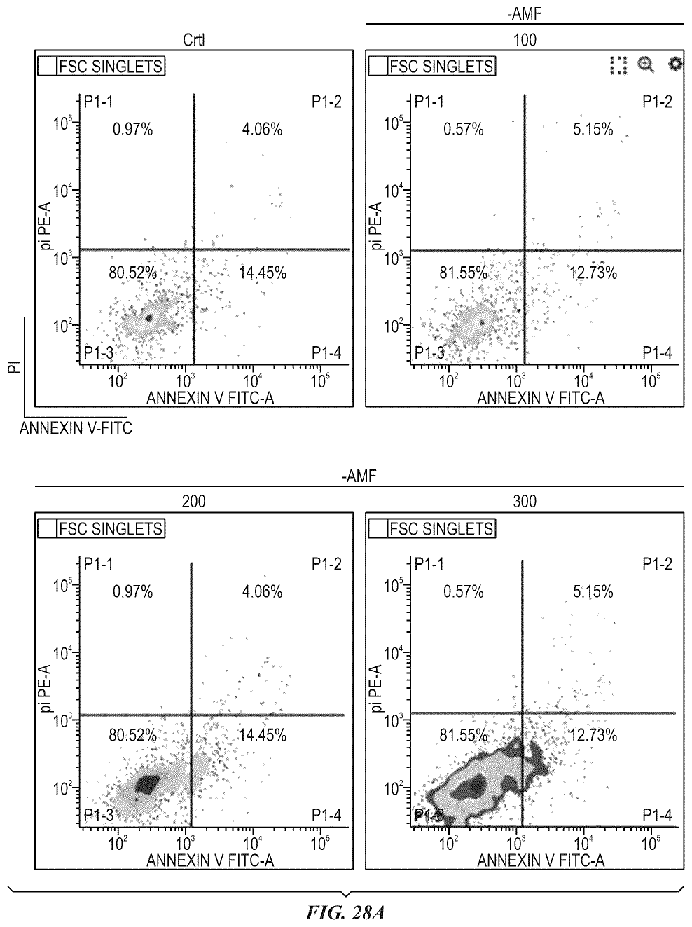

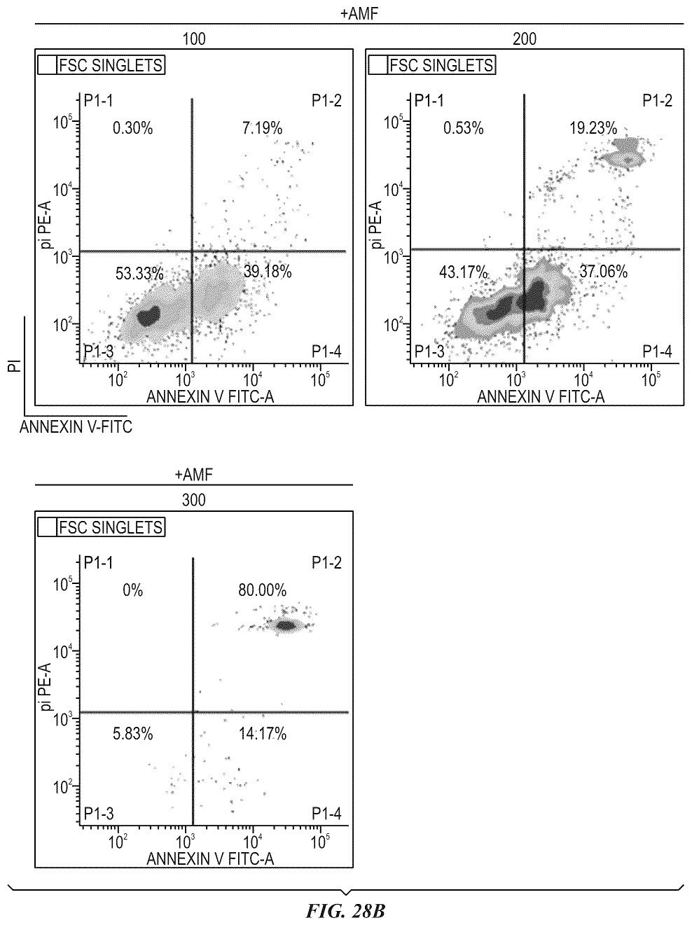

which corresponds to the lower left quadrant(s) in the plots illustrated in FIGS. 28A and 28B. As noted, under AMF, mT5 cells were killed by MIONs in a concentration-dependent manner such that:
1) with 100 μg Fe/mL of MIONs, ˜47% of cells were killed;
2) with 200 μg Fe/mL of MIONs, ˜57% of cells were killed;
3) with 300 μg Fe/mL of MIONs, ˜95% of cells were killed.

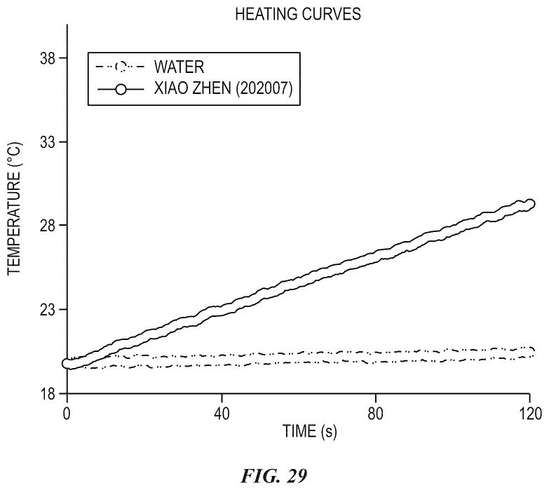

In addition, 40 nm IONCs were evaluated. Heating curves for such 40 nm IONCs are illustrated in FIG. 29. In particular, FIG. 29 illustrates the temperature increase during magnetic heating with this batch of clusters. The heating efficiency of these clusters was determined to be 333.1 W/g Fe. As discussed in the following examples, this batch of clusters was used to determine the cell killing of magnetic heating in a pancreatic cancer cell line (mT4) and release of ATP, a damage-associated molecular pattern.

In one such example the cell killing efficiency of clusters in a murine pancreatic cancer cell line, mT4 was determined. In this example, the cells were treated with 500 μg Fe/mL of MIONs under AMF for 2 h. After treatment, the cells were stained with calcein AM and Propidium Iodide (PI) to label live and dead cells, respectively. In this study, approximately 40% of the mT4 cells were killed by the treatment. The cell killing efficiency could be further increased by using this batch of clusters at a higher concentration or other batches of clusters with higher heating efficiency.

In a further example, it was determined whether treating mT4 cells with magnetic heating can induce the release of damage-associated molecular patterns, which are markers of immunogenic cell death. In this example, the mT4 cells were treated with 500 μg Fe/mL of MIONs under AMF for 2 h. Two hours after treatment, the cell culture media were collected for the quantification of ATP that was released by the cells. The concentration of ATP in the cell culture medium of mT4 cells treated with magnetic heating was significantly higher than other groups (cells only, cells under AMF, and cells with clusters but without AMF). The results indicate magnetic heating induced immunogenic cell death in mT4 cells.

In further aspects, to determine the diffusion and biodistribution of MIONs after intratumoral injection, fluorescent MIONs were prepared by labeling them with dye molecules, DiR. In this example, the dye molecules were inserted into the hydrophobic layers of the coating polymers. The fluorescent MIONs were detectable using an In Vivo Imaging System (IVIS), indicating the labeled MIONs can be used for in vivo tracking.

With this in mind, a mouse was intratumorally injected with 60 μL of fluorescent MIONs at 1.5 mg/mL. The diffusion and leakage of MIONs was examined by fluorescent imaging with IVIS. The mouse was imaged from the tumor side and the abdominal side. It was observed that the MIONs diffused to the whole tumor around one hour after the injection. The MIONs slowly leaked to the subcutaneous areas with time. The fluorescent signal in the tumor tissue was observed to be the highest (relative to other tissues) at any time point.

Signals became detectable in the liver around 2 hours after injection and increased with time. Similarly, fluorescent signals became detectable in the spleen around 3 hours after injection and increased with time. These results indicate that MIONs leaked from the tumor tissue into the circulation and accumulated in the mononuclear phagocytic system.

The organs, blood and excreta were collected for ex vivo examination of MION biodistribution. The results showed that a large amount of MIONs were retained in the tumor tissue and some MIONs accumulated in the liver and spleen, which is consistent with the in vivo imaging results.

In further studies, to determine the diffusion and biodistribution of iron oxide nanocrystal clusters (IONCs) after intratumoral injection, fluorescent IONCs were prepared by labeling them with dye molecules, Cy7. An emission spectrum of IONC-Cy7 showed that the IONCs were successfully labeled with the dye molecules. There was no fluorescence from the filtrate of the last wash, indicating the free (unconjugated) dye molecules were completely removed from the IONC-Cy7 sample. It was confirmed that the fluorescent IONCs were detectable by the In Vivo Imaging System (IVIS), indicating the labeled IONCs could be used for in vivo tracking.

A study was performed to determine how stable the fluorescence of IONC-Cy7 was after magnetic heating. In this study a mouse was intratumorally injected with IONC-Cy7 at 5 mg/mL and treated under the alternating magnetic field (AMF) for 1 hour. Before and after heating, the mouse was imaged using IVIS. It was observed via imaging that the fluorescence of IONC-Cy7 was not quenched after magnetic heating. The results indicate that the fluorescently labeled IONCs can be used to track their biodistribution after magnetic heating.

The kinetics of IONC diffusion were determined in the tumor tissue after intratumoral injection. The results showed that the IONCs diffused in the tumor tissue with time. The IONCs diffused to the entire tumor in around 2 hours.

A comparison was performed of the efficacy of IONC-based hyperthermia with or without diffusion after intratumoral injection but before heating. Two mice were used in the experiment. The first mouse was intratumorally injected with IONCs and heated under AMF for 1 hour directly after injection. The second mouse was intratumorally injected with IONCs and, after injection, the IONCs in the tumor were allowed to diffuse for 2 hours. Then the mouse was heated for 1 hour under AMF.

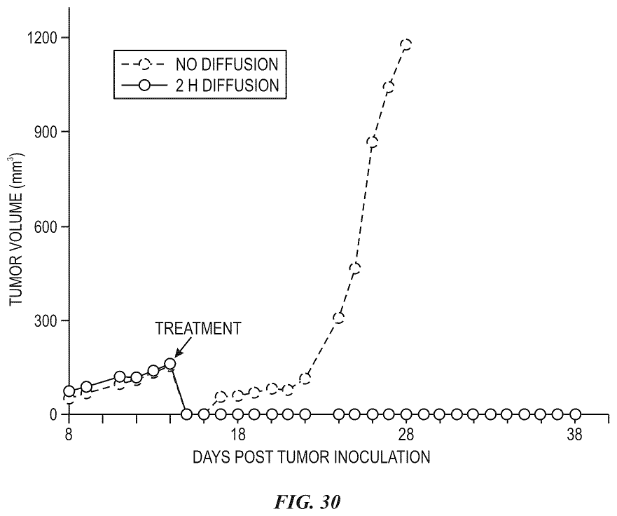

During the 1-hour heating, the maximum temperature of the tumor tissues was around 48-49° C. for both mice. The tumor sizes were measured with a digital caliper and plotted, as shown in FIG. 30. The mice were imaged 1, 7, and 14 days after the treatment.

The results showed that regardless of the diffusion, IONCs can efficiently remove most or all of the tumor tissue after 1-hour heating. With diffusion the tumor was completely eradicated by IONC-based hyperthermia. However, without diffusion, the IONCs might not be able to reach the entire tumor and the tumor cells on the edge might survive from the treatment due to insufficient heating. Tumor relapse was clearly observed 7 days post treatment and the mouse quickly reached the humane end point 14 days after treatment. In contrast, with 2-hour diffusion and 1-h heating, IONCs completely removed the subcutaneous tumor. No relapse was observed 14 days after heating.

In a further study, the fate of clusters after hyperthermia was examined. The fluorescence of IONC-Cy7 is stable in vivo, which enables long-term tracking of IONCs before and after treatment. A long-term in vivo tracking of IONCs was performed. The mouse was imaged before heating and every other day after heating. The quantification of fluorescence intensity was plotted. Acquired images showed that the fluorescent signals decreased slowly with time. The fluorescent IONCs mainly retained in the scab caused by hyperthermia and no fluorescent signals came from relapsed tumor tissue. The fluorescence dropped significantly on Day 30 because of the loss of scab tissue, further confirming that there were no IONCs in the relapsed tumor.

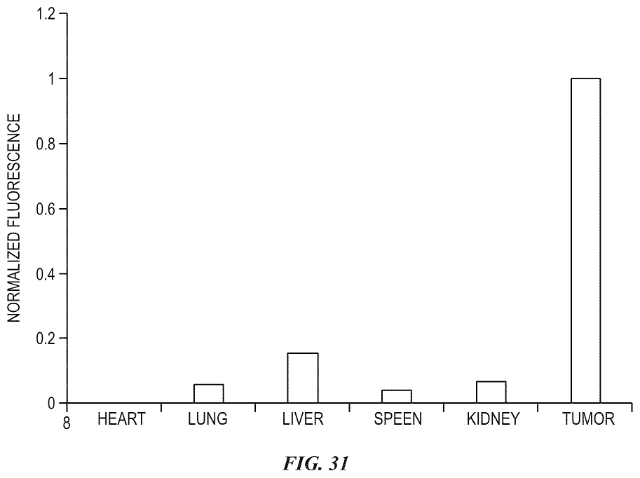

Additionally, the biodistribution of IONCs by ex vivo imaging at different time points was examined. Images showed that 24 hours after intratumoral injection of clusters, the clusters mainly retained in the tumor tissue. Weak fluorescence was detected in the liver, indicating the leaked clusters accumulated in the mouse liver. Quantification of these results is illustrated in FIG. 31. The fluorescence in lung, spleen, and kidney may be attributed to autofluorescence from these organs.

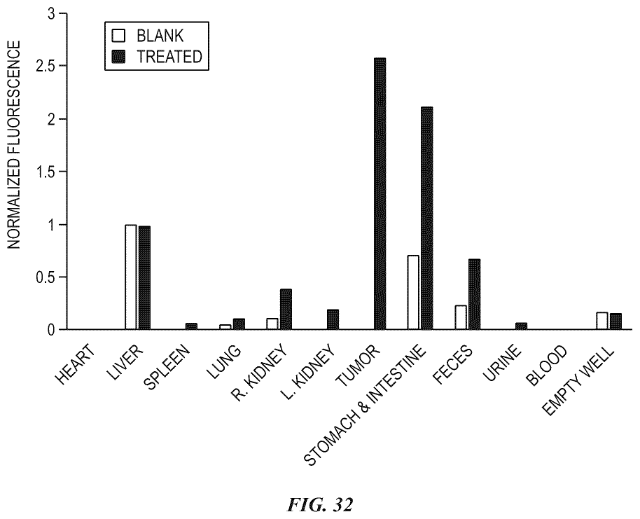

In addition, ex vivo imaging of IONC biodistribution 16 days after intratumoral injection of IONCs was investigated. Fluorescent signals were detected in the tumor, stomach, intestine, right kidney, and feces. The results indicate that the IONCs in the body might be excreted through the digestive system. Quantification of fluorescence intensity at 16 days post injection is shown in FIG. 32. The fluorescence intensity from some organs such as the liver might be a combination of organ autofluorescence and the fluorescence of IONCs. With this in mind, a blank mouse without IONC injection was included in order to compare true signals and autofluorescence in the organs, blood and excreta. As shown, for the blank mouse higher autofluorescence in liver, stomach and intestine was observed compared with other organs. The results of this comparison between mice with and without IONC injection indicate that the fluorescent signals in the tumor, stomach, intestine, right kidney, and feces are from the injection of IONCs, while the fluorescent signals in the liver are mainly autofluorescence from this organ. Moreover, there is weak fluorescence from the kidneys and the urine, which indicates the fluorescent IONCs or the dye molecules were excreted through the urinary system in addition to the digestive system.

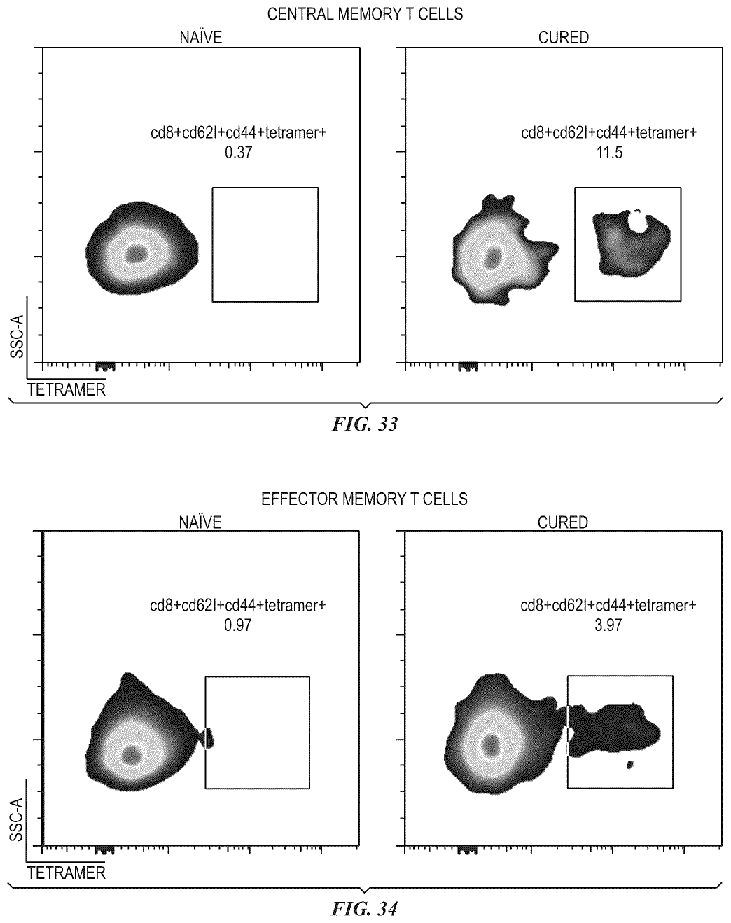

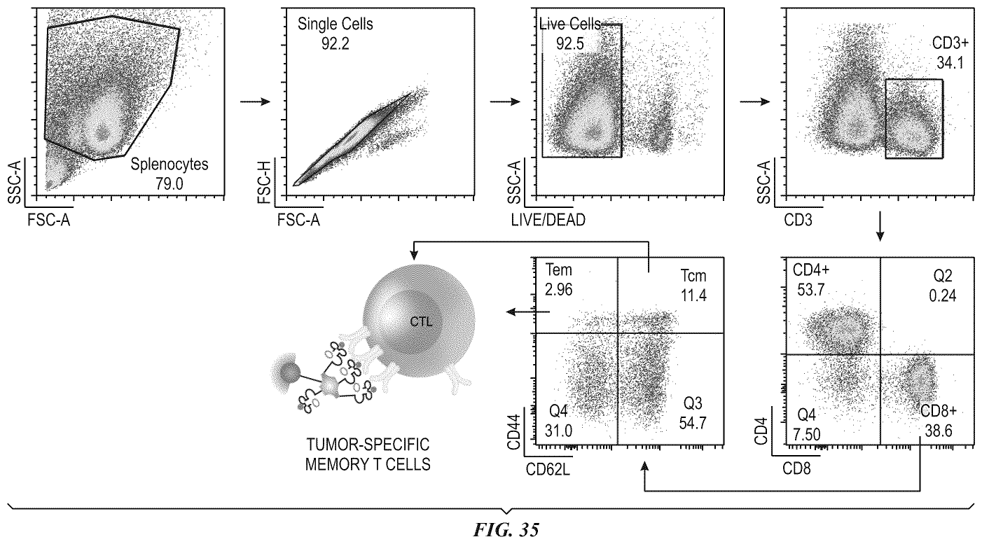

Turning to FIGS. 33 and 34, whether tumor-bearing mice cured by magneto-immunotherapy develop immune memory cell population was also investigated. The cured mice were rechallenged with the same tumor cells (MC-38 cells). As a control, mice with no previous tumor implantation were inoculated with MC-38 cells. The spleen was harvested for the analysis of immune cell subsets 10 days post tumor inoculation. Single cell suspensions were prepared from the spleen and analyzed by flow cytometry. The gating strategy is illustrated in FIG. 35. The results indicate the mice cured by magneto-immunotherapy had significantly increased tumor-specific central memory T cells (11.5% vs. 0.37% for naïve mouse) (FIG. 33) and tumor-specific effector memory T cells (3.97% vs. 0.97% for naïve mouse) (FIG. 34).

With respect to the gating strategy employed for the analysis of tumor-specific memory T cells in the preceding study, a graphical representation of the approach employed is illustrated in FIG. 35. The single cell suspensions were prepared from the spleen and stained with antibody cocktails. The single cells were first gated for live cells, and then gated for CD3+ T cells. The CD3+ T cells were then gated for CD8+ T cells. The CD3+CD8+ T cells were further divided into CD44+CD62L-(effector memory) and CD44+CD62L+ (central memory) T cells. The memory T cells were further stained with MHC-TAA tetramer for tumor specificity.

### Materials and Methods

Materials: Ethylene glycol (anhydrous, 99.8%), iron (III) chloride hexahydrate (FeCl₃·6H₂O, ACS reagent, 97%), urea (ACS reagent, 99.0%), azobis(isobutyronitrile) (AIBN, 98%), acrylic acid (anhydrous, contains 200 ppm MEHQ as inhibitor, 99%), 2-acrylamido-2-methylpropanesulfonic acid (AMPS, 99%), dimethylformamide (DMF, anhydrous, 99.8%), 3,4-dihydroxyphenethylamine hydrochloride (dopamine), poly(ethylene glycol) methyl ether methacrylate (Mw 500), poly(acrylic acid) (PAA, Mw˜1,800), sodium nitrite (ACS reagent, ≥97.0%), 2,2′-azobis(2-methylpropionamidine) dihydrochloride (AAPH) and lipid peroxidation detection kit were purchased from Sigma-Aldrich. 1-Ethyl-3-(3-(dimethylamino)-propyl)carbodiimide hydrochloride (EDC), sulfuric acid (ACS grade, 98%), hydrochloric acid (ACS grade, 37%), 2,2′-azino-bis(3-ethylbenzothiazoline-6-sulfonic acid (ABTS) solution, 2′,7′-dichlorodihydrofluorescein diacetate (H₂DCFDA), calcein acetoxymethyl ester (Calcein AM), bicinchoninic acid (BCA) protein assay kit, Alexa Fluor 568 Phalloidin, Hoechst 33342, JC-1 Dye, and Alexa Fluor 594 TUNEL assay kit were purchased from Thermo Fisher Scientific. Polyacrylic acid sodium salt (PAA, M_{w}˜6,000) was purchased from Polyscience Inc. Alexa Fluor 488 conjugated calreticulin antibody, Alexa Fluor 488 conjugated Ki-67 antibody, FITC conjugated CD3 antibody, and γ-H2AX antibody were obtained from Cell Signaling Technology. APC-conjugated CD4 antibody, PE-conjugated CD8α antibody, PE-conjugated CD11c antibody, FITC-conjugated CD80 antibody, APC-conjugated CD86 antibody, CD16/CD32 antibody, and fixable viability stain 450 were purchased from BD Biosciences. Iron staining kit, creatinine quantification kit, mouse aspartate aminotransferase (AST) ELISA kit, recombinant mouse granulocyte-macrophage colony-stimulating factor (GM-CSF), HIF-1α antibody, GAPDH antibody, FITC conjugated Hsp70 antibody, horseradish peroxidase (HRP)-conjugated goat antirabbit secondary antibody, Alexa Fluor 488 conjugated goat antirabbit antibody, and DAPI-containing mounting medium were purchased from Abcam. ATP and GSH quantification kits were obtained from Promega. The ELISA kits for quantification of mouse alanine transaminase (ALT), alkaline phosphatase (ALP), and blood urea nitrogen (BUN) were obtained from MyBioSource. OVA₂₅₇₋₂₆₄ peptide (SINFEKL) and MC-38 tumor-associated antigen peptide MuLV p15E (KSPWFTTL) were purchased from MBL. Mouse Hsp70 ELISA and mouse IFN-γ ELISpot kits were purchased from R&D Systems. Propidium iodide (Anaspec), annexin V apoptosis kit (SouthernBiotech), mycoplasma detection kit (Lonza), anti-PD-1 antibody (InvivoGen), and 4-compartment cell culture dishes (Greiner bio-one) were purchased from the indicated sources, respectively.

Synthesis of iron oxide nanocrystal clusters: Iron oxide nanocrystal clusters (IONCs) were synthesized using a solvothermal method described in the literature. Briefly, FeCl₃·6H₂O (540 mg), PAA (250 mg), urea (1200 mg), and deionized water (Milli-Q, >18 mΩ, 1.5 mL) were dissolved in ethylene glycol (20 mL) using a magnetic stirrer. This reaction mixture was vigorously stirred for 60 min, leading to a transparent yellow solution. The solution was then transferred to the Teflon-lined stainless steel autoclave reactor. The reactor was heated at 200° C. for 6 h in an oven. A black solution was obtained after cooling down the reaction solution, indicating the formation of iron oxide nanocrystal clusters. The clusters were collected using a rare earth magnet. The product was washed by a mixture of acetone and water six times to remove the byproducts and unreacted reactants. The purified clusters were redispersed in water for further use.

Replacement of PAA on cluster surface by dopamine: Five milliliters of dopamine aqueous solution (1 mg/mL) was added to 5 mL of IONC solution (1 mg Fe/mL) in a 20 mL glass vial. The mixture was stirred for 2 h. The mixture became turbid gradually during stirring, indicating the replacement of the polyacrylate by dopamine. The aggregated clusters were collected magnetically using a rare earth magnet. The sample was washed 6 times with water to remove free dopamine.

Synthesis of Poly(AA-co-AMPS-co-PEG): The copolymer was synthesized using the free radical polymerization method. Briefly, PEG-acrylate (1.0 g), AMPS (0.75 g), acrylic acid (0.25 g), and AIBN (300 mg) were dissolved in 10 mL of DMF in a 20 mL glass vial. The vial was then transferred to an oven equipped with an ultraviolet illumination lamp (LZC-4Xb photoreactor, UVA 350 nm). The mixture was magnetically stirred for 4 h under ultraviolet radiation to form polymers. The obtained polymer was purified using a dialysis bag (Cellulose Membrane, MWCO 3 kDa) to remove impurities. Then the purified copolymer was dried for 2 days using a freeze-dryer.

Grafting Poly(AA-co-AMPS-co-PEG) onto the clusters: Poly(AA-co-AMPS-co-PEG) was grafted to IONC surface through EDC conjugation under sonication. Typically, 2 mL of the copolymer solution (40 mg/mL), 1 mL of MES buffer solution (0.5 mol/L), and 1 mL of EDC solution (20 mg/mL) were added to the aggregated clusters (5 mL, 1 mg Fe/mL). The mixture of the copolymer and the clusters was then sonicated for 30 min using a probe sonicator. The poly(AA-co-AMPS-co-PEG) functionalized clusters were washed magnetically 5 times using water to remove impurities.

Synthesis of Nitrodopamine: Nitrodopamine was synthesized following a method reported in the literature. Briefly, 5 g of dopamine hydrochloride was dissolved in 150 mL of deionized water under vigorous magnetic stirring. Then 6.5 g of sodium nitrate was added to the solution, and then the mixture was cooled to 0° C. using an ice bath. Fifty milliliters of 20% sulfuric acid was added to the mixture very slowly. The ice bath was removed after the addition of sulfuric acid. The reaction mixture was stirred at room temperature overnight. Nitrodopamine hydrogensulfate was collected by filtering the reaction dispersion. The product was washed with cold water six times to remove the byproducts and impurities. The purified product was freeze-dried and stored at 4° C. for further use.

Synthesis of polymer Nitrodopamine-PAA-AAPH: The polymer of nitro-dopamine-PAA-AAPH was synthesized through EDC conjugation. Aqueous solution of PAA (0.36 mL, 50 mg/mL), MES (0.5 mL, 1 M), EDC (3.2 mL, 10 mg/mL) and 5.48 mL of deionized water were mixed under vigorous stirring. Aqueous solution of EDC (3.2 mL, 10 mg/mL) was added to the solution and the mixture was stirred vigorously at room temperature for 5 min. Aqueous solution of nitro-dopamine (2.96 mL, 2 mg/mL) was added to the solution, and the mixture was stirred for another 15 min. Then AAPH in 50 mM MES solution (10 mL, 100 mg/mL) was added to the reaction mixture. The reaction mixture was stirred for 3 more hours to attach AAPH molecules to the chain of PAA. The polymer was purified using a stirred cell (MWCO 3 kDa) to remove unreacted reactants and byproducts.

Attachment of Nitrodopamine-PAA-AAPH to IONC surface: Nitrodopamine-PAA-AAPH was attached to the cluster surface through the coordination between the functional group of catechol on nitrodopamine and iron atoms on the surface of clusters. Freshly purified nitro-dopamine-PAA-AAPH solution (10 mL, 1 mM) and IONC (10 mL, 1 mg Fe/mL) were mixed at 4° C. The mixture was mechanically shaken at 4° C. for 3 h. Then the clusters were purified using stirred cell (MWCO 500 kDa) to remove the free polymers.

Material characterizations: Transmission electron microscopy (TEM) image and high-resolution TEM (HRTEM) image of the clusters were acquired using a JEOL 2100 Field Emission Gun Transmission Electron Microscope at an acceleration voltage of 200 kV. A drop of the cluster solution was evaporated on a carbon-coated copper grid to prepare the TEM sample. The average diameter of the clusters was calculated from measurement of at least five hundred clusters. The Fourier-transform infrared (FTIR) spectra of the clusters were obtained on an IR Affinity-1S FTIR spectrometer (Shimadzu). The samples were washed eight times to remove free AAPH prior to FTIR measurement. One milliliter of a cluster solution with a concentration of 500 mg Fe/L was dropped onto the center of a glass slide. The slide was dried at 60° C. to form a thin layer of residue. The spectra were collected from 4000 to 400 cm⁻¹ at room temperature. The hydrodynamic diameter of the clusters was measured using a Wyatt Technology's Mobius dynamic light scattering instrument. The average size was obtained over three measurements for each sample. The surface area of the cluster was measured using a Quantachrome Autosorb-iQ3-MP/Kr BET Surface analyzer. Prior to the measurement, the samples were outgassed overnight under vacuum at 200° C.

Magnetic measurements: The magnetic properties of the clusters were measured using a superconducting quantum interference device (Quantum Design MPMS). The nanocrystals were dispersed in calcium sulfate at a weight ratio of approximately 1% to prevent sample movement and to reduce magnetic coupling among the nanocrystals. To calculate the mass magnetization accurately, the iron content of the samples was directly measured from the pellets after the measurements. The pellets were digested with 5 mL of 12 M hydrochloric acid, and the iron concentration of the solutions was measured by a ferrozine assay.

Cell culture: MC-38 cells were purchased from Kerafast. The cells were maintained in Dulbecco's Modified Eagle Medium (DMEM) supplemented with 10% fetal bovine serum (FBS) at 37° C. in a humidified atmosphere with 5% CO2. The cells were routinely tested using mycoplasma detection kit.

Detection of ABTS^{+·} free radicals: The generation of ABTS^{+·} free radicals was achieved via the reaction between ABTS solution and IONC-AAPH or IONC in PBS. 250 μL of ABTS solution was mixed with 250 μL of PBS containing IONC or IONC-AAPH of 300 μg Fe/mL concentration. The mixture was shielded from light and incubated at 37° C. or under an AMF (H=9.35 kA/m and ƒ=320 kHz) for 1 h. After incubation, the nanoclusters in the mixture were removed using Vivaspin centrifugal filters (MWCO 100 kDa). The absorbance of the samples containing ABTS^{+·} free radicals was measured from 400 to 950 nm with a microplate reader (Tecan Spark Multimode Microplate Reader).

Detection of intracellular free radicals: The intracellular generation of free radicals was determined using H₂DCFDA probe. Briefly, MC-38 cells were seeded in 4-compartment CELLview cell culture dishes (1×105 cells per compartment). Twenty-four hours later, the cells were incubated with IONC-AAPH or IONC at 300 μg Fe/mL for 1 h at 37° C. After incubation, the cells were exposed to AMF for 1 h. Then the cells were washed twice with PBS and stained with 2 μM of H₂DCFDA at 37° C. for 30 min. The generation of free radicals was observed using a fluorescence microscope (ZOE Fluorescent Cell Imager, Bio-Rad) or quantified via flow cytometry (BD Accuri C6 Plus).

Quantification of Intracellular Iron Oxide Nanocrystals: The intracellular iron oxide nanocrystals were quantified using a ferrozine-based colorimetric assay. MC-38 cells were incubated with IONC or IONC-AAPH at 300 μg Fe/mL for 1 h at 37° C. After incubation, the extracellular nanocrystals were removed. The cells were washed with PBS, detached with trypsin, and pelleted by centrifugation. The cell pellets were dried under vacuum and treated with 50 μL of HCl (12 M) to release intracellular iron. Then, 70 μL of NaOH (8 M), 100 μL of ammonium acetate (4 M), 100 μL of hydroxylamine HCl (5% w/w), 680 μL of water, and 1 mL of ferrozine (0.1% w/w) were added sequentially. The iron content was determined by light absorbance at 562 nm.

Examination of cell viability: Cell viability was evaluated via fluorescence imaging or flow cytometry. MC-38 cells were seeded in 4-compartment cell culture dishes and incubated overnight. To mimic hypoxic conditions, the cells were pretreated with 100 μM CoCl₂ for 24 h. Then 300 μg Fe/mL of IONC-AAPH or IONC was added to the cell culture medium. The cells were then exposed to AMF for 2 h followed by live/dead staining. The temperature of the cell culture medium was measured in real time with a fiber-optic temperature probe (Photon Control). For fluorescence imaging, the cells were costained with calcein AM (3 μM) and propidium iodide (5 μM) at 37° C. for 30 min. The images were taken using ZOE Fluorescent Cell Imager. For flow cytometry analysis, the cells were detached and stained with Annexin V Apoptosis Kit following the manufacturer's instructions. Data were collected on BD Accuri C6 Plus flow cytometer and analyzed using FlowJo software (Tree Star).

Western Blot Analysis: CoCl₂-induced hypoxia in MC-38 cells was confirmed by Western blot analysis of HIF-1α. MC-38 cells were treated with 100 μM CoCl₂ for 24 h at 37° C. The cells were lysed with RIPA buffer containing protease inhibitors (Thermo Fisher Scientific). The protein concentration of the cell lysate was quantified using BCA assay. The proteins were resolved by SDS-PAGE and transferred to a polyvinylidene fluoride (PVDF) membrane (Bio-Rad). The membrane was blocked with 5% nonfat milk and probed with HIF-1α and GAPDH antibodies followed by HRP-conjugated secondary antibodies. The target proteins were visualized using enhanced chemiluminescent HRP substrate (Thermo Fisher Scientific). The protein levels were quantified using ImageJ software.

Detection of DNA damage: The DNA damage induced by IONC-AAPH treatment was detected by γ-H2AX staining. MC-38 cells were seeded in 4-compartment cell culture dishes and cultured overnight. Then the cells were treated with 300 μg Fe/mL of IONC-AAPH or IONC at 37° C. or under AMF for 90 min. Four hours after the treatment, the cells were fixed with 4% paraformaldehyde for 20 min, permeabilized with 0.2% Triton X-100 for 20 min and blocked with 3% bovine serum albumin (BSA) for 30 min at room temperature. Then the cells were stained with an antibody against γ-H2AX at 4° C. overnight followed by staining with an Alexa Fluor 488-labeled secondary antibody for 2 h at room temperature. The nuclei were stained with Hoechst 33342 for 10 min at room temperature. The cells were imaged with a Nikon A1-Rsi confocal microscope and the fluorescence intensity of γ-H2AX was analyzed with ImageJ software.

Evaluation of F-actin morphology: The F-actin morphology of MC-38 cells was evaluated by phalloidin staining. MC-38 cells were seeded in 4-compartment cell culture dishes and cultured overnight. The cells were then treated with 300 μg Fe/mL of IONC-AAPH or IONC at 37° C. or under AMF for 90 min. Four hours after the treatment, the cells were fixed, permeabilized, and blocked as described above. Then the cells were incubated with Alexa Fluor 568-labeled phalloidin for 30 min at room temperature, followed by staining with Hoechst 33342 for 10 min at room temperature. Images were collected using a Nikon Al-Rsi confocal microscope.

Determination of mitochondrial health: The mitochondrial membrane potential of MC-38 cells was determined with JC-1 staining assay. MC-38 cells cultured in 4-compartment cell culture dishes were treated with 300 μg Fe/mL of IONC-AAPH or IONC at 37° C. or under AMF for 90 min. Four hours post the treatment, the cells were stained with JC-1 dye at 10 μg/mL at 37° C. for 20 min. After staining, the cells were imaged with a confocal microscope (Nikon A1-Rsi confocal) or analyzed on a flow cytometer (BD Accuri C6 Plus). The ratio of J-aggregates to J-monomers was calculated using the red and green fluorescence intensity measured by flow cytometry.

Quantification of intracellular GSH: MC-38 cells were seeded in 4-compartment cell culture dishes at a density of 1×10⁵ cells per compartment and cultured overnight. Then the cells were treated with 300 μg Fe/mL of IONC-AAPH or IONC at 37° C. or under AMF for 2 h. Two hours after the treatment, the culture medium was removed and the intracellular GSH was quantified using a luminescence-based GSH-Glo Glutathione Assay according to the manufacturer's instructions.

Determination of lipid peroxidation: The lipid peroxidation of MC-38 cells was determined by measuring the production of malondialdehyde (MDA). MC-38 cells cultured in 4-compartment cell culture dishes were treated with 300 μg Fe/mL of IONC-AAPH or IONC at 37° C. or under AMF for 2 h. Two hours post the treatment, the cells were lysed, and the MDA levels were quantified using a fluorescence-based MDA Assay Kit following the manufacturer's instructions.

Detection of damage-associated molecular patterns: Damage-associated molecular patterns (DAMPs), including calreticulin (CRT), heat shock protein 70 (Hsp70), and adenosine triphosphate (ATP), were analyzed at different time points after the treatment. Briefly, MC-38 cells were seeded in 4-compartment cell culture dishes at a density of 1×10⁵ cells per compartment and cultured overnight. Then the cells were treated with 300 μg Fe/mL of IONC-AAPH or IONC at 37° C. or under AMF for 2 h. To detect CRT and Hsp70 exposure on cell surface, the cells were detached 2 and 6 h after the treatment and stained with fluorophore-conjugated anti-CRT or anti-Hsp70 antibody on ice for 30 min. Then the cells were analyzed by flow cytometry. To determine ATP release induced by the treatment, the cell culture medium was collected 2 and 6 h post the treatment. The extracellular ATP was quantified using an ATP bioluminescence detection kit following the manufacturer's instructions. To determine the change in Hsp70 expression, the cells were lysed 24 h post the treatment. The expression of Hsp70 was quantified by ELISA and normalized to total protein.

Determination of Dendritic Cell Maturation: The bone marrow-derived dendritic cells (BMDCs) were prepared from the bone marrow of 8-week old C57BL/6N mice according to an established protocol. To determine the stimulatory effect on BMDCs, IONC-AAPH-treated MC-38 cells (together with the medium containing DAMPs) were cocultured with BMDCs for 24 h in a transwell system (pore size=3 μm). BMDCs only (without coculture) were included as the blank groups. The BMDCs were collected, blocked with CD16/CD32 antibody, and stained with PECD11c, FITC-CD80, and APC-CD86. The data were acquired on MA900 Multi-Application Cell Sorter (Sony) and analyzed using FlowJo software (Tree Star).

Tumor killing effect of IONC-AAPH in vivo: All animal work was approved by the Institutional Animal Care and Use Committee (IACUC) of Rice University. C57BL/6N mice (4-6 weeks, female) were purchased from Charles River Laboratories. The direct tumor killing effect of IONC-AAPH treatment was evaluated with subcutaneous tumor-bearing mice. Briefly, 5×10⁵ MC-38 cells were subcutaneously injected into the right flank of the mice. The mice were randomly divided into 6 groups (n=3): saline, AAPH, IONC, IONC-AAPH, IONC+AMF, and IONC-AAPH+AMF. When the tumor volume reached 150-200 mm³, the mice were administrated with saline, AAPH (2 mM), IONC (5 mg Fe/mL), or IONC-AAPH (5 mg Fc/mL) through intratumoral injection at a speed of 3 μL/min using a syringe pump (World Precision Instruments). The injection volume was 0.3 μL per mm³ tumor tissue. The mice in IONC+AMF and IONC-AAPH+AMF groups were treated with AMF (H=9.35 kA/m and ƒ=320 kHz) for 1 h. A customized polycarbonate cradle with a heating pad was placed underneath the mice to maintain the body temperature during anesthesia. The temperature in the tumor was measured using a high-resolution infrared (IR) camera (E95, Teledyne FLIR). The tumor size was measured daily with a digital caliper, and the tumor volume was calculated as follow: volume=length×width²/2.

Twenty-four hours after the treatment, the tumor tissues were collected for H&E, Ki-67, and TUNEL staining. At the end of the study, blood samples were collected for quantification of ALT, AST, ALP, BUN, and creatinine. The tumors were excised, weighed, and photographed. The major organs, including heart, lung, liver, spleen, and kidney, were harvested and examined by H&E staining. These organs together with the tumor-draining and the contralateral nondraining inguinal lymph nodes were examined for iron distribution by Prussian blue iron staining.

Abscopal effect of Mag-IT: The abscopal effect was determined with mice bearing bilateral subcutaneous tumors. 5×10⁵ and 1×10⁵ MC-38 cells were injected into the right and left flanks of the mice. The tumor on the right side was designated as the primary tumor for IONC-AAPH treatment, and the tumor on the left side was designated as the secondary (distant) tumor without IONC-AAPH treatment. The mice were randomly divided into 6 groups (n=8): saline, anti-PD-1, IONC-AAPH, IONC-AAPH+anti-PD-1, IONC-AAPH+AMF, and IONC-AAPH+AMF+anti-PD-1. When the volume of the primary tumor reached 50-100 mm³, saline or IONC-AAPH (7.5 mg Fe/mL) was injected directly into the primary tumor using the syringe pump. The injection volume was 0.3 μL per mm3 tumor tissue. The mice in IONC-AAPH+AMF and IONC-AAPH+AMF+anti-PD-1 groups were treated with AMF for 1 h. 1, 4, and 7 days after the treatment, the mice in IONC-AAPH+AMF+anti-PD-1 group were administrated with anti-PD-1 antibody (10 mg/kg) through intraperitoneal (IP) injection. The anti-PD-1 antibody was also administrated to the mice in anti-PD-1 and IONC-AAPH+anti-PD-1 groups following the same schedule. The tumor sizes were measured daily with a digital caliper, and the tumor volume was calculated as follows: volume=length×width²/2. For survival analysis, the mice were euthanized when the tumors reached the maximum permitted size (15 mm in any dimension) and counted as dead.

Flow Cytometry Analysis of Immune Cells in Untreated Distant Tumors: The tumor implantation and treatments were performed as described above. On Day 17 (10 days after the first treatment), the distant tumors were collected for flow cytometry analysis of tumor infiltrating lymphocytes. Briefly, the tumor tissue was minced into small pieces and digested with 300 U/mL collagenase, 100 U/mL hyaluronidase, and 0.15 mg/mL DNase I (Stem Cell Technology) at 37° C. for 30 min under gentle shaking. The tumor tissue was then transferred to a 70 μm nylon mesh strainer to remove large pieces of undigested tissue. The cells filtered through the strainer were treated with ammonium chloride solution to remove red blood cells. Then the single-cell suspensions were incubated with anti-CD16/CD32 to block nonspecific binding to Fc receptors. The cells were further stained with the viability dye and fluorophore-conjugated antibodies against CD3 (FITC), CD4 (APC), and CD8 (PE). The data were acquired on MA900 Multi-Application Cell Sorter (Sony) and analyzed using FlowJo software (Tree Star).

IFN-γ ELISpot Assay: The tumor implantation and treatments were performed as described above. On Day 17 (10 days after the first treatment), the mouse spleens were harvested from different treatment groups for the preparation of single-cell suspensions. The splenocytes (2×10⁵ cells per well) were seeded into a 96-well plate precoated with anti-IFN-γ antibody. The cells were incubated with or without SINFEKL (OVA peptide) or KSPWFTTL (tumor-associated antigen peptide) at 10 μg/mL for 24 h at 37° C. The ELISpot assay was performed using the Mouse IFN-γ ELISpot Kit (R&D Systems) according to the manufacturer's instructions. The IFN-γ spots were counted manually under a stereomicroscope.

Immune memory effect: The immune memory effect was investigated by rechallenging the surviving mice with MC-38 cells. Briefly, 5×10⁵ MC-38 cells were first transplanted into the right flank of the mice. The mice were randomly divided into 2 groups: IONC-AAPH+AMF and IONC-AAPH+AMF+anti-PD-1. When the tumor volume reached 50-100 mm3, the tumors were injected with IONC-AAPH (7.5 mg/mL) and treated with AMF for 1 h. The treatment was repeated once if the tumor was not completely removed by the first round of IONC-AAPH treatment. The mice in the IONC-AAPH+AMF+anti-PD-1 group were administrated with anti-PD-1 antibody (10 mg/kg of body weight) 1, 4, and 7 days after the first round of IONC-AAPH treatment. 40 days after the first tumor was removed, the mice were rechallenged by transplanting 5×10⁵ MC-38 cells into the left flank. The tumor growth was monitored daily. A group of naive mice (without previous tumor implant) were transplanted with 5×10⁵ MC-38 cells in the left flank for comparison of tumor growth rate.

Statistical analysis: All data are presented as mean ±s.d. or mean ±s.e.m. Statistical analysis was performed using GraphPad Prism (v8.0). Statistical tests are indicated in the figure legends. P values are indicated by asterisks in the figures as *P<0.05, **P<0.01, ***P<0.001, and ****P<0.0001. P<0.05 was considered statistically significant.

### Technical Effects and Conclusions

The present disclosure is directed to a combination cancer therapy that integrates magnetic hyperthermia, heat-triggered free radicals, and immune checkpoint blockade (ICB) therapy into a single treatment modality. In certain embodiments, a nanoplatform consisting of one or more IONCs and one or more AAPH molecules generates localized heat and free radicals upon AMF actuation. The simultaneous generation of heat and free radicals from IONC-AAPH effectively killed tumor cells through causing oxidative stress and damaging multiple cellular components, including DNA, actin cytoskeleton, and mitochondria. Magnetic heating and free radicals synergistically evoked the exposure of calreticulin, the release of ATP, and the upregulation of HSP70, thereby dramatically increasing the immunogenicity of the tumor cells. This treatment modality successfully eradicated primary tumors, inhibited distant tumors through the abscopal effect, and induced long-term immune memory against tumor rechallenge. The combination of IONC-AAPH-based magnetotherapy with ICB therapy may help control cancer recurrence and metastasis and improve the response of ICB therapy in clinical settings.

In summary, the presently described techniques relate to a magneto-immunotherapy for solid tumors that utilizes magnetic iron oxide nanocluster (IONC) based heat and free radical generation with immune checkpoint blockade therapy. Upon applying an alternating magnetic field, the IONCs produce a high level of local heat, decomposing the attached AAPH molecules, resulting in carbon-centered free radicals. The simultaneous generation of heat and free radicals from IONC-AAPH effectively killed tumor cells by causing intracellular GSH depletion and damaging multiple cellular components including DNA, actin cytoskeleton, mitochondria, and lipid membranes. The tumor cell death caused by combined magnetic heating and free radicals is highly immunogenic, as demonstrated by cell surface translocation of CRT and Hsp70, and release of ATP, which promoted dendritic cell maturation. Treating the primary tumors with IONC-AAPH under AMF led to the eradication of the tumors. Further, the combination of IONC-AAPH under AMF with anti-PD-1 ICB dramatically inhibited the growth of untreated distant tumors by inducing tumor-specific T cell response and increasing tumor-infiltrating CD8⁺ T cells. In addition, this magneto-immunotherapy also induced a strong long-term immune memory effect against tumor rechallenge. Hence, the IONC-AAPH-based magneto-immunotherapy has the potential to effectively control cancer recurrence and combat cancer metastasis, thus significantly improving the current cancer therapies.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method, comprising:
positioning a nanoplatform within a region adjacent to tumor tissue of a subject, wherein the nanoplatform comprises one or more iron oxide nanocrystal clusters (IONCs) and one or more 2,2′Azobis (2-midinopropane) dihydrochloride (AAPH) molecules; and
applying, via a magnetic field generator, a magnetic field to the region adjacent to the tumor tissue.

2. The method of claim 1, wherein the magnetic field is applied to the region over a first time period, and wherein the method comprises performing a quantification of the tumor tissue after applying the magnetic field.

3. The method of claim 2, comprising applying, via the magnetic field generator, the magnetic field to the region over a second time period based on the quantification of the tumor tissue.

4. The method of claim 1, wherein the region adjacent to the tumor tissue of the subject is a subcutaneous region.

5. The method of claim 1, wherein the magnetic field is an alternating magnetic field.

6. A method, comprising:
positioning a nanoplatform within a subcutaneous region of a subject, wherein the subcutaneous region includes tumor tissue of the subject, and wherein the nanoplatform comprises one or more iron oxide nanocrystal clusters (IONCs) and one or more 2,2′Azobis (2-midinopropane) dihydrochloride (AAPH) molecules;
applying, via a magnetic field generator, a magnetic field to the subcutaneous region including the tumor tissue; and
administering an immune checkpoint blockade (ICB) therapy treatment to the subject.

7. The method of claim 6, wherein the magnetic field is applied to the subcutaneous region during a first time period, and the ICB therapy treatment is administered during a second time period.

8. The method of claim 7, wherein the first time period substantially overlaps with the second time period.

9. The method of claim 6, comprising determining a quantitative baseline associated with a size of the tumor tissue before applying the magnetic field.

10. The method of claim 9, comprising determining a change in the size of the tumor tissue by comparing a measurement of the tumor tissue after applying the magnetic field to the quantitative baseline.

11. A method, comprising:
administering a magnetotherapy treatment to tumor tissue of a subject, wherein the magnetotherapy treatment comprises positioning a nanoplatform adjacent to or in contact with the tumor tissue and applying a magnetic field to the nanoplatform; and
administering an immune checkpoint blockade (ICB) treatment to the subject.

12. The method of claim 11, wherein the nanoplatform comprises one or more iron oxide nanocrystal clusters (IONCs) and one or more 2,2′Azobis (2-midinopropane) dihydrochloride (AAPH) molecules.

13. The method of claim 11, comprising measuring a quantity of cell death of the tumor tissue after administering the magnetotherapy treatment to the tumor tissue.

14. The method of claim 13, wherein measuring the quantity of cell death of the tumor tissue comprises determining a concentration of calreticulin (CRT), heat shock protein 70 (HSP70), adenosine triphosphate (ATP), or a combination thereof, in a region comprising the tumor tissue.

15. The method of claim 11, wherein the magnetotherapy treatment is administered over a first time period and the ICB treatment is administered over a second time period, and wherein the first time period overlaps with the second time period.

16. A nanoplatform, comprising:
a metal oxide cluster; and
one or more free radical generators disposed on a surface of the metal oxide cluster.

17. The nanoplatform of claim 16, wherein the metal oxide cluster comprises one or more iron oxide nanocrystal clusters (IONCs).

18. The nanoplatform of claim 16, wherein the one or more free radical generators comprise one or more 2,2′Azobis (2-midinopropane) dihydrochloride (AAPH) molecules.

19. The nanoplatform of claim 16, wherein the one or more free radical generators are attached to the surface of the metal oxide cluster via a nitrodopamine-poly(acrylic acid) chain.

20. The nanoplatform of claim 16, wherein the nanoplatform generates heat and one or more free radicals in response to an interaction with a magnetic field.

21. A nanoplatform, comprising:
a magnetic oxide cluster; and
one or more free radical generators attached to a surface of the magnetic oxide cluster;
wherein the nanoplatform simultaneously generates heat and one or more free radicals in response to an interaction with a magnetic field.

22. The nanoplatform of claim 21, wherein the one or more free radical generators are bonded to the surface of the magnetic oxide cluster via a polymer chain.

23. The nanoplatform of claim 21, wherein the magnetic oxide cluster comprises one or more iron oxide nanocrystal clusters (IONCs).

24. The nanoplatform of claim 21, wherein the one or more free radical generators comprise one or more 2,2′Azobis (2-midinopropane) dihydrochloride (AAPH) molecules.

25. A method of preparing the nanoplatform, comprising:
synthesizing a metal oxide cluster via metal salt hydrolysis; and
bonding one or more free radical generators to a surface of the metal oxide cluster.

26. The method of claim 25, wherein the metal oxide cluster comprises one or more iron oxide nanocrystal clusters (IONCs).

27. The method of claim 25, wherein the one or more free radical generators comprise one or more 2,2′Azobis (2-midinopropane) dihydrochloride (AAPH) molecules.

28. The method of claim 25, comprising coating a surface of the metal oxide cluster with polyacrylic acid (PAA).

29. The method of claim 28, comprising modifying the surface of metal oxide cluster with poly(AA-co-AMPS-co-PEG).

30. The method of claim 25, wherein the one or more free radical generators are bonded to the surface of the metal oxide cluster via a nitrodopamine-PAA chain.
